(19) United States
(12) Patent Application Publication
(10) Pub. No.: US 2023/0044633 A1
(43) Pub. Date: Feb. 9, 2023

(54) OPTICAL BASED METHODS FOR DETERMINING ANTIMICROBIAL DOSING REGIMENS

(71) Applicant: University of Houston System, Houston, TX (US)

(72) Inventors: Michael NIKOLAOU, Houston, TX (US); Vincent H. TAM, Bellaire, TX (US); Iordanis KESISOGLOU, Houston, TX (US)

(21) Appl. No.: 17/636,079

(22) PCT Filed: Aug. 21, 2020
(86) PCT No.: PCT/US2020/047408

§ 371 Date: Feb. 17, 2022

Related U.S. Application Data
(60) Provisional application No. 62/889,807, filed on Aug. 21, 2019.

(51) Int. Cl. G16H 20/10; G16H 50/50

(52) U.S. Cl. CPC G16H 20/10; G16H 50/50

(57) ABSTRACT

An optical based method determines the most clinically effective antimicrobial agent treatment for a subject afflicted with a microbial infection, including those subjects that have developed resistance to said microbial agents. The provided method is based on the ability to discriminate between live and dead microbes growing in a culture medium.

### TECHNICAL FIELD

The present disclosure relates to an optical based method for determining a clinically effective antimicrobial agent treatment for a subject afflicted with a microbial infection, including cases where those microbes have developed resistance to one or more antimicrobial agents. The provided method is based on the ability to distinguish between live and dead microbes in a population of microbes in a culture medium and exposed to one or more antimicrobial agents, with the microbial population size of live and dead cells combined continuously monitored by an optical based method. The provided method permits one to determine the rate of microbe killing induced by one or more antimicrobial agents, including development of microbial resistance to such agents, continuously over time.

### BACKGROUND

The alarming spread of antimicrobial resistance is threatening our antimicrobial armamentarium (Arias, 2009, N Eng J Med. 360:439-43). In the U.S., nearly 2 million people acquire bacterial infections while in the hospital and 90,000 of these individuals die annually (Klevens, 2007, Pub Health Rep. 122:160-6). The total cost of antimicrobial resistance to U.S. society is a staggering $5 billion each year (Institute of Medicine, 1998). P. aeruginosa, A. baumannii and K. pneumoniae are commonly implicated in serious nosocomial infections such as pneumonia and sepsis; they are also associated with multiple mechanisms of resistance to various antibiotics (efflux pumps, ß-lactamase production, porin channel deletion, target site mutation, etc.) (Bonomo, 2006, Clin. Infect. Dis. 1:43 Suppl. 2:S49-56; Landman, 2009, Epid Biol Infect 137:174-80; Livermore, 2002, Clinical Infectious Diseases 34:634-640; Urban, 1994, Lancet 344:1329-32). The treatment of these (multi-)drug resistant infections represents a challenge to clinicians, as many (if not all) available antibiotics are ineffective and infections due to these pathogens have been shown to be associated with unfavorable clinical outcomes (Cao, 2004, Mol Microbiol 53:1423-36; Harris, 1999, Clin. Infect Dis 28:1128-33; Kwa, 2007 Antimicrob Agents chemotherapy 54; 3717-3722; Kwa et al 2007, Antimicrob Agents chemotherapy 54; 1160-4; Tam, 2010b Diag Microbiol Inf Dis 58:99-104). As a result, a concerted effort is urgently needed to develop effective treatments to combat these infections (Talbot, 2006, Clin Infect Dis 42:657-68). However, new antimicrobial agents take time to develop and are unlikely to be available in time to solve this crisis (Cooper, 2011, Nature 472:32).

As a last resort, clinicians often turn to combinations of existing antibiotics to treat infections due to these problematic pathogens. However, design of combination therapy is presently poorly guided. When selecting combination therapy to treat an infected patient, a clinician currently lacks the information to make a rational decision and the time to investigate bacterial resistance mechanisms. Specifically, the possible permutations of various variables to consider for the design of combination therapy (e.g., agent(s), dose, dosing frequency, duration of IV administration) make prohibitive the comprehensive evaluation of all possibilities. As a result, most clinical decisions for combination therapy are made empirically, based on either anecdotal experience or intuition. Accordingly, methods for aiding clinicians in design of combination therapies are greatly needed.

### SUMMARY

The present disclosure is directed to rapid methods for determining the response of a microbial cell population to one or more antimicrobial agent treatments. The methods are based on the use of optical signaling to detect the response of a microbial cell population, in contact with one or more antimicrobial agents, over time. A previous disadvantage of using optical signals to estimate viable bacterial burden was the inability to distinguish live from dead cells. The present disclosure provides methods for mathematically handling the inability to experimentally distinguish between live and dead cells, thereby providing a more accurate determination of microbial cell growth or decline in the presence of antimicrobial agents. Such methods provide individualized and effective treatment strategies for microbe-infected subjects, including those subjects that have been infected by microbes that have developed resistance to antimicrobial agents.

The provided methods include exposing a microbial cell population to one or more of a series of fixed concentrations of one or more antimicrobial agents over time and measuring changes in the microbial population in the presence of the antimicrobial agent. Changes in the microbial cell population are measured through the detection of optical signals that measure changes in microbe density over time. The following mathematical framework (1) includes equations, which when supplied with input data, i.e., measurements of the size of the total population of (live and dead) bacterial cells over time, are able to predict microbial response of live cells to one or more antimicrobial agents:

$$\{ \begin{matrix}\frac{{dN}_{total}}{dt} = ( {K}_{g} [ 1 - \frac{{N}_{live} ( t )}{{N}_{m a x}} ] + {K}_{d} ) {N}_{live} ( t ) \\ \frac{{dN}_{live}}{dt} = ( {K}_{g} [ 1 - \frac{{N}_{live} ( t )}{{N}_{m a x}} ] - {r}_{m i n} - λ {ae}^{- at} ) {N}_{live} ( t )\end{matrix} \} \qquad ( 1 )$$

where Nₜₒₜₐₗ is the total bacterial population; Nₗᵢᵥₑ is the bacterial population that is alive; Nₘₐₓ is the maximum bacterial population; K₉ is the growth rate constant; K_{d} is the natural death rate constant; rₘᵢₙ is the kill rate of the most resistant sub-population; λ is the magnitude of adaptation; and a is the rate of adaptation.

The present disclosure provides a method for determining a clinical dosing regimen that is pharmacologically effective against a microbial cell population in an infected subject based on the values over the time period of the mathematical framework (1) above. The present disclosure is also directed to a method of treating a subject having a pathological condition caused by infection with a microbial cell population using the determined dosing regimen. In another aspect, the present disclosure is directed to a method of preventing a pathological condition in a subject having been exposed to a microbial cell population using the determined dosing regimen.

Such dosing regimens may include administration of a single antimicrobial agent or combinations of one or more antimicrobial agents over a given treatment time.

The present disclosure is directed further to a method for suppressing emergence of acquired resistance of a microbial cell population to one or more antimicrobial agents useful for treating a pathological condition associated therewith in a subject. The method includes administering to the subject a pharmacologically effective amount of the one or more antimicrobial agents on a dosing regimen determined via the mathematical framework (1).

In yet another aspect, a method is provided including screening one or more potential antimicrobial agents, alone or in combination, for efficacy in treating and/or suppressing resistance acquisition in one or more cell populations using the provided mathematical framework (1). In another aspect, a method is provided that relates to compiling a library of antimicrobial agents and dosing regimens effective to treat and/or suppress the emergence of acquired resistance in microbial cell populations.

The provided methods further include exposing a microbial cell population to one or more of a series of fixed concentrations of one or more antimicrobial agents over time and measuring changes in the microbial population in the presence of the antimicrobial agent. Changes in the microbial cell population are measured through the detection of optical signals that measure changes in microbial cell density over time. The following mathematical framework (2), which results from analytical solution of equations (1), when supplied with input data, i.e., measured changes in total (live and dead) microbial cell population size over time, is able to fit said input data by estimation of corresponding parameters:

$$\frac{{N}_{total} ( t )}{{N}_{0}} = {e}^{λ ( {e}^{- at} - 1 ) + ( {K}_{g} - {r}_{m i n} ) t} ++ \\ {e}^{- λ} {λ}^{\frac{{K}_{g} - {r}_{m i n}}{a}} ( \frac{{K}_{d} + {r}_{m i n}}{a} {\int }_{λ {e}^{- at}}^{λ} {z}^{- 1 + \frac{{r}_{m i n} - {K}_{g}}{a}} {e}^{z} dz + {\int }_{λ {e}^{- at}}^{λ} {z}^{\frac{{r}_{m i n} - {K}_{g}}{a}} {e}^{z} dz ) \qquad ( 2 )$$

where Nₜₒₜₐₗ, Nₗᵢᵥₑ, Nₘₐₓ, K₉, K_{d}, rₘᵢₙ, λ and a are as previously described. The estimated values of said parameters can then be used in to integrate forward in time the second differential equations of (1) to predict the response of live microbial cells to one or more antimicrobial agents.

The present disclosure provides a method for determining a clinical dosing regimen that is pharmacologically effective against a microbial cell population in an infected subject based on the values over the time period of the mathematical framework (2) above. The present disclosure is also directed to a method of treating a subject having a pathological condition caused by infection with a microbial cell population using the determined dosing regimen. In another aspect, the present disclosure is directed to a method of preventing a pathological condition in a subject having been exposed to a microbial cell population using the determined dosing regimen.

Such dosing regimens may include administration of a single antimicrobial agent or combinations of one or more antimicrobial agents over a given treatment time.

The present disclosure is directed further to a method for suppressing emergence of acquired resistance of a microbial cell population to one or more antimicrobial agents useful for treating a pathological condition associated therewith in a subject. The method includes administering to the subject a pharmacologically effective amount of the one or more antimicrobial agents on a dosing regimen determined via the mathematical framework (2).

In yet another aspect, a method is provided including screening one or more potential antimicrobial agents, alone or in combination, for efficacy in treating and/or suppressing resistance acquisition in one or more cell populations using the provided mathematical framework (2). In another aspect, a method is provided that relates to compiling a library of antimicrobial agents and dosing regimens effective to treat and/or suppress the emergence of acquired resistance in microbial cell populations.

### BRIEF DESCRIPTION OF THE DRAWINGS

Various embodiment of the present methods are described herein with reference to the drawings wherein:

FIG. 1. Theoretical and actual model performance. Left: decline followed by regrowth manifested as a lag in growth—viable counts (Nₗᵢᵥₑ) in dashed line and anticipated optical signals (N_{measured}) in continuous line. Right: a typical model fit to a bacterial growth profile captured by Bacterioscan 216Dx.

FIG. 2. The correlation of two fluctuating profiles to an array of concentrations. Left: Pharmacokinetic profiles of two drugs (depicted by continuous and dashed lines) with different elimination half-lives and dosing frequencies. Right: A factorial concentration array is used to emulate representative concentration combinations over time; A: high concentrations of both drugs; E: high concentration of 1 drug and low concentration of the other; B and D: high concentration of 1 drug and intermediate concentrations of the other; C and F: intermediate concentration of one drug and low concentration of the other; G: low concentrations of both drugs; Ctrl: no drug (control).

FIG. 3. A. baumannii 1261 was exposed to 16 different concentration combinations of levofloxacin (L) and amikacin (A). Each sequence of points represents the time course of the bacterial population (baseline inoculum approximately 2-5×10⁵ CFU/ml) exposed to one levofloxacin/amikacin concentration combination (e.g., levofloxacin 20 mg/l+amikacin 30 mg/ is shown in hollow squares). Bacteria exposed to drugs with no activity produce profiles that superimpose on those from placebo controls; antimicrobial activity manifests as a delay or absence of growth.

FIG. 4. Qualitative effect of an antibiotic at a time invariant concentration on a heterogeneous bacterial population comprising subpopulations of varying degrees of antibiotic resistance. As the antibiotic concentration is set at increasingly higher values, the bacterial response over time changes from full growth to the point of saturation (in the absence of antibiotic), to retarded growth, to regrowth (resulting from rapid decline of bacterial subpopulations highly susceptible to the antibiotic combined with growth of subpopulations less susceptible to the antibiotic), then retarded regrowth, and finally complete eradication of the entire bacterial population. Complete eradication will not occur if a resistant subpopulation is included in the original bacterial population or developed in the course of antibiotic exposure.

FIG. 5. Qualitative patterns in measurements of total number of (live and dead) bacterial cells (thick lines) corresponding to populations of live bacterial cells (thin lines) over time, in response to time invariant antibiotic concentrations, as described in FIG. 4.

FIG. 6. Typical profiles for each of eqns. (15), (16) and (17).

FIG. 7 Fit of Eqn. 3 to experimental data on Nₗᵢᵥₑ generated by plating for a bacterial population of AB exposed to LVX at a number of time invariant concentrations.

FIG. 8. Comparison of experimental data produced by the optical density instrument to the output of Eqns. (2) and (B1), with parameter values set at the averages of the three estimates produced from the data fits reported in Table 1, referring to FIG. 7.

FIG. 9. Fit of eqn. (2) to experimental data on Nₜₒₜₐₗ generated by the optical density instrument for a bacterial population of AB exposed to LVX at a number of time invariant concentrations.

FIG. 10. Fit of eqn. (2) to experimental data on Nₜₒₜₐₗ generated by the optical density instrument for a bacterial population of AB exposed to LVX at a number of time invariant concentrations for 24 h.

FIG. 11. Fit of eqn. (2) to experimental data on Nₜₒₜₐₗ generated by the optical density instrument for a bacterial population of AB exposed to LVX at a number of time invariant concentrations for 12 h.

FIG. 12. Fit of eqn. (2) to experimental data on Nₜₒₜₐₗ generated by the optical density instrument for a bacterial population of AB exposed to LVX at a number of time invariant concentrations for 9 h.

FIG. 13. Fit of eqn. (2) to experimental data on Nₜₒₜₐₗ generated by the optical density instrument for a bacterial population of AB exposed to LVX at a number of time invariant concentrations for 6 h.

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

As used herein, the term “subject” refers to a mammal, in some cases a human, that is the recipient of an antimicrobial for treatment or prevention of a pathological condition associated with a microbial population.

As used herein, the term “cell population” refers to a microbial cell population.

As used herein, the term “microbe” generally refers to multi-cellular or single-celled organisms and include, for example, bacteria, protozoa, and fungi. Microbes include, but are not limited to, all of gram-negative (Gram −) and gram-positive (Gram +) bacteria, Eumycetes, Archimycetes, and so forth. In non-limiting examples, the microbe may be at least one microbe selected from Enterococcus, Streptococcus, Pseudomonas, Salmonella, Escherichia coli, Staphylococcus, Lactococcus, Lactobacillus, Enterobacteriacae, Klebsiella, Providencia, Proteus, Morganella, Acinetobacter, Burkholderia, Stenotrophomonas, Alcaligenes, and Mycobacterium. The microbe may also include Enterococcus faecium, Staphylococcus aureus, Klebsiella species, Acinetobacter baumannii, Pseudomonas aeruginosa, and Enterobacter species, but example embodiments of the concepts described herein are not limited thereto.

As used herein, the term “antimicrobial agent” refers generally to an agent that kills a microbe, stops or slows a microbe's growth. Such antimicrobials include, but are not limited to, Amikacin, Amoxicillin, Ampicillin, Aztreonam, Benzylpenicillin, Clavulanic Acid, Cefazolin, Cefepime, Cefotaxime, Cefotetan, Cefoxitin, Cefpodoxime, Ceftazidime, Ceftriaxone, Cefuroxime, Ciprofloxacin, Dalfopristin, Doripenem, Daptomycin, Ertapenem, Erythromycin, Gentamicin, Imipenem, Levofloxacin, Linezolid, Meropenem, Minocycline, Moxifloxacin, Nitrofurantoin, Norfloxacin, Piperacillin, Quinupristin, Rifampicin, Streptomycin, Sulbactam, Sulfamethoxazole, Telithromycin, Tetracycline, Ticarcillin, Tigecycline, Tobramycin, Trimethoprim, and Vancomycin.

In embodiments, the present disclosure provides a method for determining the response of a microbial population to one or more antimicrobial agents over time, including: exposing the microbial population to a series of fixed concentrations of one or more antimicrobial agents over time; determining rates of change of the antimicrobial cell population growth over time in the presence of the one or more antimicrobial agents; and imputing said data into the following mathematical model or modeling framework:

$$( 1 ) \{ \begin{matrix}\frac{{dN}_{total}}{dt} = ( {K}_{g} [ 1 - \frac{{N}_{live} ( t )}{{N}_{m a x}} ] + {K}_{d} ) {N}_{live} ( t ) \\ \frac{{dN}_{live}}{dt} = ( {K}_{g} [ 1 - \frac{{N}_{live} ( t )}{{N}_{m a x}} ] - {r}_{m i n} - λ {ae}^{- at} ) {N}_{live} ( t )\end{matrix} \} \qquad ( 1 )$$

where Nₜₒₜₐₗ is the total bacterial population; Nₗᵢᵥₑ is the bacterial population that is alive; Nₘₐₓ is the maximum bacterial population; K_{g} is the growth rate constant; K_{d} is the death rate constant; rₘᵢₙ is the kill rate of the most resistant sub-population; λ is the magnitude of adaptation; and a is the rate of adaptation.

In other embodiments, the present disclosure provides a method for determining the response of a microbial population to one or more antimicrobial agents over time, including: exposing the microbial population to a series of fixed concentrations of one or more antimicrobial agents over time; determining rates of change of the antimicrobial cell population growth over time in the presence of the one or more antimicrobial agents; and imputing said data into the following mathematical model or modeling framework (2):

$$\frac{{N}_{total} ( t )}{{N}_{0}} = {e}^{λ ( {e}^{- at} - 1 ) + ( {K}_{g} - {r}_{m i n} ) t} ++ \\ {e}^{- λ} {λ}^{\frac{{K}_{g} - {r}_{m i n}}{a}} ( \frac{{K}_{d} + {r}_{m i n}}{a} {\int }_{λ {e}^{- at}}^{λ} {z}^{- 1 + \frac{{r}_{m i n} - {K}_{g}}{a}} {e}^{z} dz + {\int }_{λ {e}^{- at}}^{λ} {z}^{\frac{{r}_{m i n} - {K}_{g}}{a}} {e}^{z} dz ) \qquad ( 2 )$$

where Nₜₒₜₐₗ, Nₗᵢᵥₑ, Nₘₐₓ, K_{g}, K_{d}, rₘᵢₙ, λ, and a are as previously described. Mathematical equations related to the framework predicting bacterial response to various drug exposures are attached as Appendix A, B and C.
For determining the rates of changes in growth of a microbial cell population, any optically based instrument or device may be used that provides real-time quantification of microbial cell population growth. In a non-limiting embodiment, a BacterioScan 216Dx laser microbial growth monitor (BacterioScan, Inc.) can be used to rapidly measure microbe cell population densities with high precision. The BacterioScan platform relies on measurement of both optical density and forward-angle laser light-scattering of suspended particles in liquid samples. A laser beam is passed through custom-made disposable cuvettes, and the angular distribution of scattered laser light is captured on a charge coupled device (CCD) camera located at the opposite end of the cuvette. The resulting raw signals are fed into a proprietary data integration algorithm, which translates the inputs into colony forming units per milliliter (CFU/ml) values. Since the instrument can incubate samples at an optimal temperature for, for example, bacterial growth (35-37° C.), taking repeated measurements over time allows for particle number expansion or stasis to be visualized. These measures correlate with microbe resistance or susceptibility, respectively, when microbe cells are incubated in the presence of different antibiotics.

An advantage associated with the use of the BacterioScan 216Dx platform is its ability to track a microbe population in real-time. The microbe response during antibiotic exposure can be monitored as frequently as every 5 minutes over an extended timeframe (e.g., 4-48 hours). These information-rich datasets may then be used as input data for the mathematical framework disclosed herein to predict microbe eradication or outgrowth in an extended timeframe (up to days in a course of therapy).

The present disclosure provides a method for determining a clinical dosing regimen that is pharmacologically effective against a microbial cell population in a subject based on the values over the time period addressed by the mathematical modeling framework (1) and/or (2). The method includes (i) collecting information-rich datasets that indicate microbial cell population growth response in the presence of one or more antimicrobial agents over a period of time; (ii) inputting the datasets into the mathematical modeling framework (1) and/or (2) for determining the susceptibility of the microbial cell population during contact with the one or more antimicrobial agents over the period of time; (iii) correlating, at the end of the time period, an increase in microbe susceptibility in the presence of the antimicrobial agent with a likely clinical dosing regimen that is pharmacologically effective against a microbial cell population in a subject. The method further provides for administration of said antimicrobial agent in the determined doses.

Adaptation of a microbial population when exposed to fixed concentrations of an antimicrobial agent can be captured in a form of a mathematical modeling framework and its associated parameter estimates. The mathematical modeling framework captures the relationship between microbial burden and antimicrobial agent concentrations. The mathematical modeling framework enables a method for guiding highly targeted testing of dosing regimens, which could substantially accelerate development of antimicrobial agents. More particularly, standard time-kill studies data over 24 hours are used as framework inputs. The utility of a large number of dosing regimens can be effectively screened in a comprehensive fashion, where promising regimens are investigated further in pre-clinical studies and clinical trials. It is contemplated that because the dosing regimens are designed to prevent resistance emergence, the clinical utility lifespan of new antimicrobial agents or drugs would be prolonged.

The present disclosure is also directed to a method of treating a subject having a pathological condition caused by infection with a microbial cell population using the determined dosing regimen. In such an instance, the one or more tested antimicrobial agents are administered to the subject at the determined dosing regimen.

In another aspect, the present disclosure is directed to a method of preventing a pathological condition in a subject arising from exposure to a microbial cell population using the determined dosing regimen. In such an instance, the one or more tested antimicrobial agents are administered to the exposed subject at the determined dosing regimen.

The present disclosure provides a method for determining a clinical dosing regimen that is pharmacologically effective against a microbial cell population that has developed a resistance to one or more antimicrobial agents in a subject based on the values over the time period of the mathematical framework (1) and/or (2) as above. The method includes: (i) collecting information-rich datasets that indicate microbial cell population response in the presence of one or more antimicrobial agents over a period of time wherein the microbial cell population has developed resistance to one or more antimicrobial agents; (ii) inputting the datasets into the mathematical modeling framework (1) and/or (2) for determining the susceptibility of the microbial cell population during contact with the one or more antimicrobial agents over the period of time; (iii) generating an output value of the susceptibility of the microbe cell population based on the mathematical modeling framework; and/or (iv) based on the generated output value, correlating at the end of the time period, an increase in microbe susceptibility in the presence of the one or more antimicrobial agents with a likely clinical dosing regimen that is pharmacologically effective against a resistant microbial cell population in a subject.

The present disclosure is directed further to a method for suppressing emergence of acquired resistance of a microbial cell population to an antimicrobial agent useful for treating a pathological condition associated with an infected subject. The method includes administering to the subject a pharmacologically effective amount of an antimicrobial agent on a dosing regimen determined via the disclosed mathematical framework (1) and/or (2) of growth response over a period of time that the microbial cell population is in contact with the antimicrobial agent.

In another aspect, a method is provided including screening a potential antimicrobial agent for efficacy in treating and/or suppressing resistance acquisition in one or more cell populations using the provided mathematical modeling framework (1) and/or (2). In yet another embodiment, a method is provided for high-throughput screening for antimicrobial agents effective to suppress emergence of acquired resistance thereto in a cell population associated with a pathophysiological condition, including: inputting values utilizing the mathematical modeling framework (1) and/or (2) having equations for calculating over a specified time period, a rate of change of cellular susceptibility to the antimicrobial agent and a rate of change of cell burden in a surviving cell population, said equations operably linked to the initial parameter values; and correlating, at or near the end of the time period, an increase in cellular susceptibility output values and a decrease in cell population growth values with suppression of emergence of acquired resistance within the cell population to the antimicrobial agent.

Further to this embodiment, the method may include compiling a library of antimicrobial agents and dosing regimens effective to suppress the emergence of acquired resistance in cell populations. In both embodiments, the initial parameter values may correspond to time, infusion rate of the antimicrobial, volume of distribution, clearance of the antimicrobial, concentration to achieve 50% of maximal kill rate of a cell population, and maximum size of a cell population and constants for maximum adaptation and adaptation rate of a cell population and growth rate, maximum kill rate and sigmoidicity of a cell population.

The present disclosure provides dosing regimens that are pharmacologically effective against a microbial population based on the output values over the time period of the mathematical modeling framework (1) and/or (2). The dosing regimens may be used to treat or prevent in a subject a pathological condition caused by the microbial population for which the dosing regimen was designed.

In embodiments, the microbial cell population may be a population of Gram-negative bacteria, Gram-positive bacteria, yeast, mold, mycobacteria, virus, or various infectious agents. Representative examples of Gram-negative bacteria are Escherichia coli, Klebsiella pneumoniae, Pseudomonas aeruginosa and Acinetobacter baumannii. Representative examples of Gram-positive bacteria are Streptococcus pneumoniae and Staphylococcus aureus. In some embodiments, the microbial cell population is a S. aureus. S. epidermidis, E. faecalis or E. aerogenes infection. A representative example of a virus is HIV or avian influenza. The pathophysiological conditions may be any such condition associated with or caused by a microbial population. Particularly, the pathophysiological condition may be a nosocomial infection.

In some embodiments, the infection is caused by a methicillin-resistant or vancomycin-resistant pathogen. In some embodiments, the infection is a methicillin-resistant S. aureus (MRSA) infection. In some embodiments, the infection is a quinolone-resistant S. aureus (QRSA) infection. In some embodiments, the infection is a vancomycin-resistant S. aureus (VRSA) infection.

Antimicrobial agents for use in the treatment of a subject may include anti-bacterials, antifungals and/or antivirals. Routes of administration of an antimicrobial agent and pharmaceutical compositions, formulations and carriers thereof are standard and well known in the art. They are routinely selected by one of ordinary skill in the art based on, inter alia, the type and status of the pathological condition, whether administration is for antimicrobial or prophylactic treatment, and the subject's medical and family history.

As can be appreciated, the devices and/or systems can include, or be operably coupled to any suitable computing device, circuitry, and/or controllers to receive, analyze, and/or communicate information or data (e.g., via electrical signals) As used herein, the term “controller” and like terms are used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device. The term “controller” also includes “processor,” “digital processing device” and like terms, and are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some embodiments, the operating system is provided by cloud computing.

In some embodiments, the controller includes a storage and/or memory device. The storage and/or memory device is one or more physical apparatus used to store data or programs on a temporary or permanent basis. In some embodiments, the controller includes volatile memory and requires power to maintain stored information. In some embodiments, the controller includes non-volatile memory and retains stored information when it is not powered. In some embodiments, the non-volatile memory includes flash memory. In some embodiments, the non-volatile memory includes dynamic random-access memory (DRAM). In some embodiments, the non-volatile memory includes ferroelectric random access memory (FRAM). In some embodiments, the non-volatile memory includes phase-change random access memory (PRAM). In some embodiments, the controller is a storage device including, by way of non-limiting examples, CD-ROMs, DVDs, flash memory devices, magnetic disk drives, magnetic tapes drives, optical disk drives, and cloud computing based storage. In some embodiments, the storage and/or memory device is a combination of devices such as those disclosed herein.

In some embodiments, the controller includes a display to send visual information to a user. In some embodiments, the display is a cathode ray tube (CRT). In some embodiments, the display is a liquid crystal display (LCD). In some embodiments, the display is a thin film transistor liquid crystal display (TFT-LCD). In some embodiments, the display is an organic light emitting diode (OLED) display. In various some embodiments, on OLED display is a passive-matrix OLED (PMOLED) or active-matrix OLED (AMOLED) display. In some embodiments, the display is a plasma display. In some embodiments, the display is a video projector. In some embodiments, the display is interactive (e.g., having a touch screen or a sensor such as a camera, a 3D sensor, a LiDAR, a radar, etc.) that can detect user interactions/gestures/responses and the like. In still some embodiments, the display is a combination of devices such as those disclosed herein.

As can be appreciated, the controller may include or be coupled to a server and/or a network. As used herein, the term “server” includes “computer server,” “central server,” “main server,” and like terms to indicate a computer or device on a network that manages the disclosed devices, components, and/or, resources thereof. As used herein, the term “network” can include any network technology including, for instance, a cellular data network, a wired network, a fiber optic network, a satellite network, and/or an IEEE 802.11a/b/g/n/ac wireless network, among others.

In some embodiments, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In embodiments, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed devices and/or systems.

The systems described herein may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

### Example 1

Traditional testing of antibiotic combinations (e.g., checkerboard method and time-kill studies) is for the most part based on results at the end of the observation period. These methods are labor-intensive and the results have not been well correlated to clinical outcomes (Hilf, 1989, Am J Med 87:540-6; Saballs, 2006, J Antimicrob Chemother 58:697-700). Over the years, several mathematical modeling frameworks have been established that aim to make accurate predictions of the bacterial response to clinically relevant concentrations of antibiotics, which can fluctuate over time (Bhagunde, 2010, Antimicrob Agents Chemother 54:4739-43; Bhagunde, 2011, J Antimicrob Chemother 66:1079-86; Nikolaou, 2006a J Math Biol 52:154-82; Nikolaou, 2007, Ann Biomed Eng 35:1458-70). While these frameworks have been validated for several antibiotics against different bacteria, the clinical application of these methods remains limited in view of the requirement for longitudinal data input—i.e., data that capture how bacteria respond to antibiotics over short time scales and across different regimens. Instead of relying on microbiological methods to quantify bacterial burden, an imaging-based approach is used to capture such data, providing a breakthrough in technical capabilities. For example, the BacterioScan automated microbiology platform may be used for this purpose, providing the starting point to investigate how combined antibiotic activity can be harnessed to combat drug resistance.

Laboratory (e.g., ATCC) and clinical isolates of P. aeruginosa, A. baumannii and K. pneumoniae are studied (up to 20 isolates each species) for their response to antibiotic treatment. These three species of Gram-negative bacteria are commonly encountered in hospital-acquired infections. The susceptibility of the isolates to a screening panel of antibiotics is determined to ascertain their wild-type or multidrug-resistant phenotypes. The clonal relatedness of the isolates is assessed by pulsed-field gel electrophoresis; clonally-unique isolates are used whenever possible to enhance the generalizability of the approach.

Six antibiotics, a representative member of each major antibiotic family (e.g., meropenem, levofloxacin, amikacin, rifampin, minocycline and polymyxin B) are used for testing. All six agents are currently used to treat clinical (Gram-negative) infections. From the development standpoint, using antibiotics from different structural classes also enhances the robustness of the technical platform.

To ensure the BacterioScan optimal signals provide accurate CFU/ml quantifications, suspensions of representative strains from each of the three pathogens to be studied is prepared in different growth media (e.g., Mueller Hinton Broth with or without cation supplementation), and serial 10-fold dilutions of each suspension are made into fresh medium. At least one antibiotic susceptible and one multidrug-resistant clinical isolate is evaluated for each target pathogen. Suspensions of different inocula (e.g., 10³⁻⁷ CFU/ml) are analyzed in real time, with corresponding plate-based measurements being made hourly until the stationary phase is reached. Following regulatory (FDA) performance criteria for a medical device, instrument reproducibility is evaluated to identify any sources of variability between instruments; replicate suspensions are prepared and loaded into different units and run concurrently. To assess day-to-day variability (bias and precision), these assays will be repeated with freshly prepared suspensions on 5-6 consecutive days. A successful representation of changes in bacterial density over time is achieved when ≤10% variation between instrument output and plate-based CFU/ml values are observed.

Despite the capacity of data acquisition, one disadvantage of using optical signals to estimate viable bacterial burden is the inability to distinguish live from dead cells. The physical attributes (e.g., scattered light) captured by the spectroscopic imaging approach are influenced by live, non-growing or dead cells. Signals from actively growing bacterial populations are more information-rich than those from declining populations. In view of this limitation, a mathematical modeling framework (1) has been identified to account for this potential drawback. In embodiments, bacterial population can be eradicated if and only if rₘᵢₙ is greater than K_{g}.

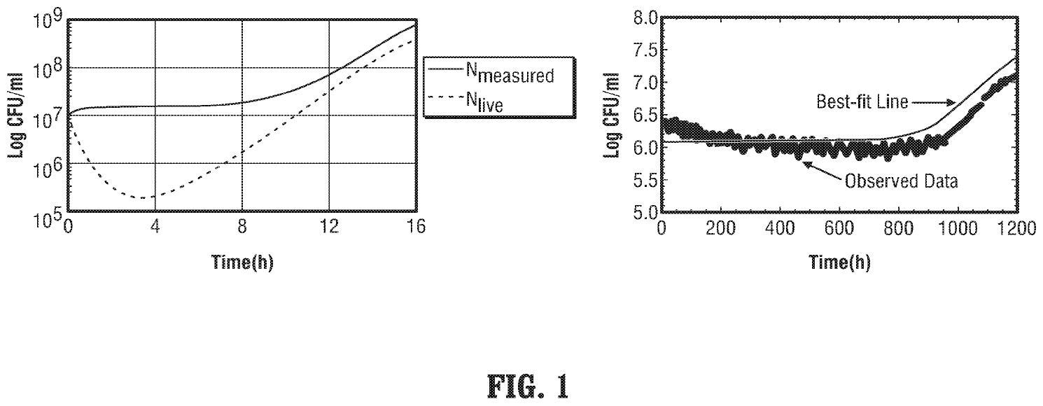

Under this framework, bacterial regrowth after an initial decline is manifested as a delay in growth (FIG. 1).

Many antibiotics are ineffective alone against multidrug resistant bacteria, but some antibiotics may have improved antibacterial activity when used in combination. Since multidrug resistance can be mediated by various molecular mechanisms, determining these useful combinations for a patient-specific isolate can be labor intensive. Thus, devising a simpler method to identify antibiotic combinations that are effective against multidrug resistant bacteria would have significant antimicrobial implications. Traditional approaches to evaluating combined antibiotic activity are associated with implicit assumptions and do not correlate with clinical outcomes. More robust modeling approaches have been proposed, but the application of these tools in a clinical setting is challenging.

A method for screening useful antibiotic combinations against multidrug resistant bacteria has been validated (Hirsch, 2013, J Infec Dis 207:786-93; Lim, 2008, Antimicrob Agents Chemother 52:2898-904; Yuan, 2010, J Infec Dis 201: 889-97). This framework is the starting point for the development of a tool for patient-specific antibiotic combination selection.

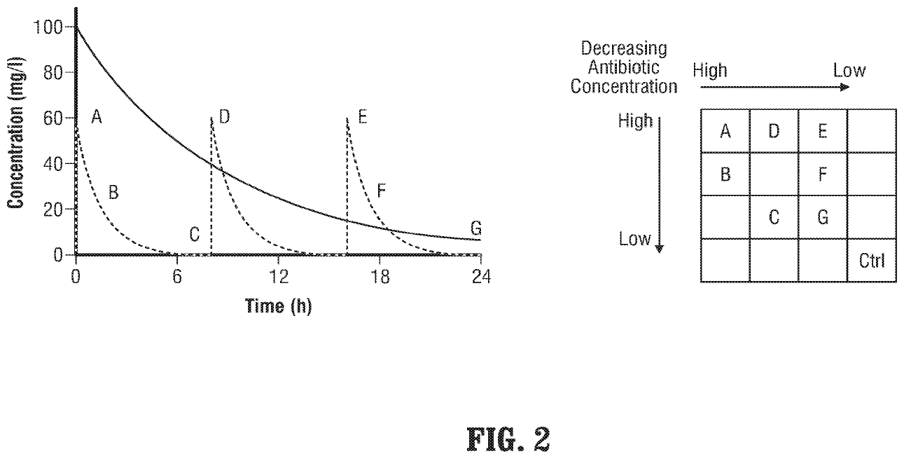

Combinations of different drug concentrations correlate better to clinical drug exposures with fluctuating concentration profiles. When antibiotics are given to patients, the serum drug concentrations fluctuate over time. However, almost all in vitro testing methods rely on fixed drug concentrations. To improve the ability to infer in vivo outcomes from in vitro testing, a factorial concentration array can be used (FIG. 2). Kill rates derived from different concentration combinations can be integrated using mathematical modeling as described below to achieve more reliable predictions of treatment outcomes.

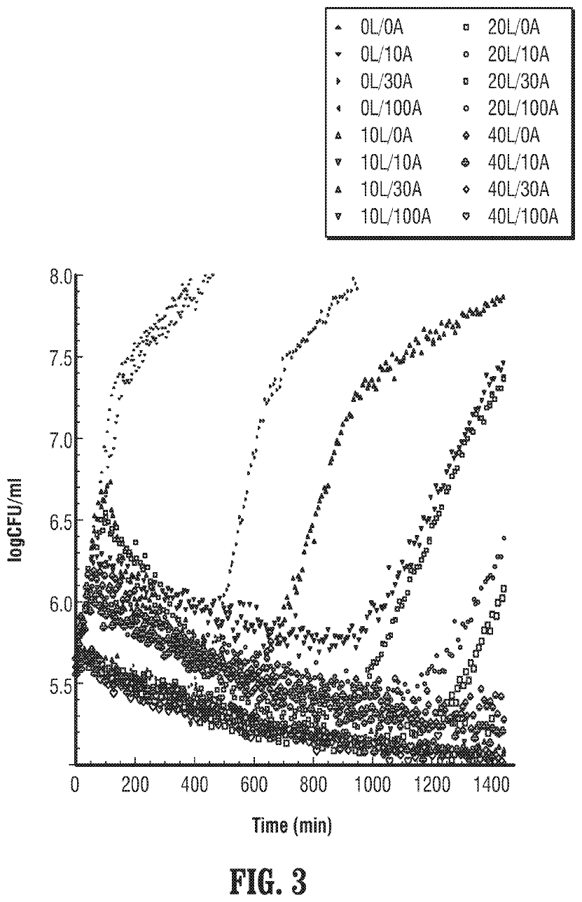

Ineffective antibiotics may have acceptable activity when combined. A clinical isolate of A. baumannii was exposed to different antibiotic concentration combinations in a 4×4 array, and the bacterial burden was tracked over 24 h using the prototype technical platform 216Dx. While the isolate is resistant to levofloxacin, amikacin and cefepime (multidrug resistant), bacterial growth was delayed in selected combinations (FIG. 3) and the profiles were reasonably captured by the mathematical modeling framework (1) shown above (FIG. 1, right).

A modeling framework for quantifying the intensity of antibiotic dosing regimens. A mathematical modeling framework has been developed that employs standard time-kill data to predict the effect of single-antibiotic dosing regimens on bacterial populations with varying degrees of resistance (U.S. Pat. No. 8,452,543; “Sidebar 1 Equations (2) and (3)”). This framework has been validated for several antibiotics against different bacteria (Tam, 2011).

Sidebar 1—Dynamics of heterogeneous bacterial populations (Nikolaou, 2006b)
Starting with the balance dN/dt=(K_{g}−r)N(t) for a homogeneous population, a heterogeneous population (with bacteria of varying degrees of resistance) has been shown to satisfy the equations:

$$\frac{dN ( t )}{dt} = [ K \text{?} - μ ( t ) ] N ( t ) \text{?} \frac{d μ ( t )}{dt} = - {σ ( t )}^{2} , \{ \frac{dk \text{?} ( t )}{dt} = - \text{?} ( t ) \} \text{?} ( \text{?} = μ \text{?} = {σ}^{2} ) \qquad ( 2 )$$

$$\text{?} \text{indicates text missing or illegible when filed}$$

where N(t)=bacterial population size at time t; K_{g}=growth rate constant; μ(t), σ²(t)=average and variance of the drug-concentration dependent kill rate constant r distributed over the populations, respectively; κₙ(t)=n-order cumulant of r(C) (Weisstein, 2005] and A is the adaptation rate constant. Simplifying (Poisson-like) assumptions yield:

$$ln [ \frac{N ( t )}{{N}_{0}} ] = ( K \text{?} μ ( 0 ) + \frac{{σ ( 0 )}^{2}}{A} ) t + \frac{{σ ( 0 )}^{2}}{{A}^{2}} ( c \text{?} 1 ) , μ ( t ) \text{?} μ ( 0 ) = \frac{{σ ( 0 )}^{2}}{A} + \frac{{σ ( 0 )}^{2}}{A} e \text{?} \qquad ( 3 )$$

$$\text{?} \text{indicates text missing or illegible when filed}$$

The modeling framework expresses the dosing intensity (D) of different antibiotic dosing regimens, regardless of the concentration/time-dependency of bacterial killing. The framework relies on an index D/K_{g} together with explicit formulas for its calculation (Nikolaou, 2007). Using longitudinal bacterial response data as inputs, parameters in equation (3) are fit, and a surface is plotted for D/K_{g} as a function of dosing regimens (daily dose and dosing interval) for related host pharmacokinetics. Effective combination regimens are those in which the combined kill rate exceeds bacterial growth rate (corresponds to D/K_{g}>1) resulting in bacterial suppression. Predictions of combined antimicrobial activity for different two-agent combinations were subsequently validated in a neutropenic murine pneumonia model.

Selection of multidrug resistant bacteria. Up to five clinical and multidrug-resistant isolates from each of P. aeruginosa, A. baumannii and K. pneumoniae are studied. The specific mechanism(s) conferring multidrug resistance (e.g., ß-lactamase production, target site alteration and efflux pump over-expression) are determined. Clonally-unique isolates (with different mechanisms of resistance) are used if possible.

Selection of antibiotics. Six antibiotics as detailed above are used. Fifteen two-agent combinations are tested by selecting two antibiotics from different structural classes.

Animals. Swiss Webster mice (male and female, 21-25 grams) will be allowed to eat and drink ad libitum.

Comparing the activity of different antibiotic combinations. In vitro studies are performed to generate data on the activity of different antibiotics against the above-mentioned bacteria. The studies employ an increasing concentration of any two of the six antibiotics in a n×n array, as guided by the optimal conditions (e.g., growth media, initial inoculum, etc.) determined as above. The bacterial populations are monitored every 5 minutes for up to 72 hours, as shown in FIG. 3.

Development of a predictive model. To ensure that the input data from patient-specific bacteria can be optimally used in a clinical setting, the mathematical model is modified by considering time to an endpoint (e.g., 1-log increase) (Sidebar 2: “Sidebar 2 equation (4)”).

Sidebar 2. Novel characterization of drug interaction (Tam, 2004)
The combined effect of two antibiotics on a bacterial population is characterized through the equation:

$${\underbrace{T \text{?}}}_{\begin{matrix}Theoretical time - to - endpoint \\ of drugs A \& B\end{matrix}} = f ( {\underbrace{T \text{?}}}_{\begin{matrix}Time - to - endpoint \\ i n absence of drug\end{matrix}} , {\underbrace{T \text{?} ( C \text{?} )}}_{\begin{matrix}Time - to - endpoint \\ for drug A\end{matrix}} , {\underbrace{T \text{?} ( C \text{?} )}}_{\begin{matrix}Time - to - endpoint \\ for drug B\end{matrix}} ) \qquad ( 4 )$$

$$\text{?} \text{indicates text missing or illegible when filed}$$

where T_{AB} is the combined effect of drugs A and B; T_{intercept} is the time to endpoint in the absence of drug; T_{A}(C_{A}) and T_{B}(C_{B}) represent the time-to-endpoint as a function concentration of individual drugs A and B, respectively; and the function ƒ(T_{intercept}, T_{A}(C_{A}), T_{B}(C_{B})) refers to the theoretical time to endpoint that would result from the combined use of non-interacting drugs A and B, at concentrations C_{A} and C_{B}. An analytical expression for ƒ can be obtained, where the average, μ(0), and variance σ(0)² of the kill rate constant resulting from the combined use of drugs A and B at t=0 is assumed to be additive.

A response surface (where x and y-axes represent different antibiotic concentrations and the z-axis depicts the time to endpoint) is used to describe the anticipated effect under various constant concentration combinations.

The effect expected from a fluctuating concentration-time profile is projected by integrating responses observed from various concentration combinations in the factorial array (FIG. 2, right). A time-based interaction index is derived by comparing the anticipated time to endpoint to the observed time, in order to describe the nature and extent of the pharmacodynamic interaction between the two antibiotics investigated.

### Example 2

Determining the pharmacodynamics of an infectious bacterial population exposed to antibiotics in vitro can provide guidance towards the design of effective therapies for challenging clinical infections. However, accomplishing this task by conducting detailed time-kill experiments is resource-limited, therefore typically bypassed in favor of empirical shortcuts. The resource limitation could be addressed by continuously assessing the size of a bacterial population under antibiotic exposure using optical density measurements over time. However, such measurements count both live and dead cells and, while usable with growing bacterial populations, they cannot assess the size of a declining population of live cells. The present disclosure fills this void by providing a model-based method that uses combined counts of both live and dead cells to infer the number of live cells in a bacterial population exposed to antibiotics. Thus, the in vitro pharmacodynamics of the interaction between bacterial population and antibiotics can be easily discerned, and therapy can be guided. The method is general enough for populations comprising bacteria of varying degrees of susceptibility to one or multiple antibiotics, makes no assumptions about the underlying mechanisms that confer resistance, and is applicable to any microbial population whose monitoring, under exposure to antimicrobial agents, yields combined counts of live and dead cells. The example below, demonstrates the use of a model-based method in an experimental study on the response of Acinetobacter baumannii exposed to levofloxacin as described below.

While time kill experiments combined with plating for assessment of bacterial population size are a standard research tool, they are time-consuming, labor-intensive, and produce a limited number of data points. This makes them difficult to use in situations where time or resources are limited yet reliable results are needed quickly, e.g. in a clinical setting. A substantially more efficient alternative to plating would be continuous assessment of the size of a bacterial population in suspension. Measurements of sample turbidity (cloudiness) by optical density methods (spectrophotometry) can fulfill that requirement (Mytilinaios et al., (2012) International Journal of Food Microbiology 154:169-176; Lopez et al., (2004) International Journal of Food Microbiology 96: 289-300; McMeekin et al., (1993) Predictive Microbiology: Theory and Application. Wiley, New York). Optical density measurements rely on well known principles and can easily provide a continuous stream of data in real time.

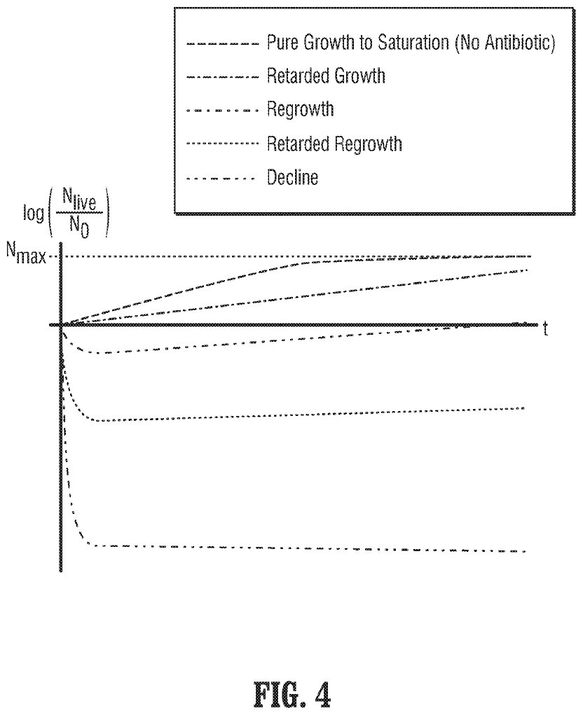

However, optical density measurements also have a basic limitation: They count both live and dead cells in a bacterial population, as both kinds of cells produce an optical signal by blocking/absorbing light. Therefore, optical density measurements are typically suitable for monitoring a growing bacterial population, but cannot keep track of a declining population that exhibits patterns as shown in FIG. 4.

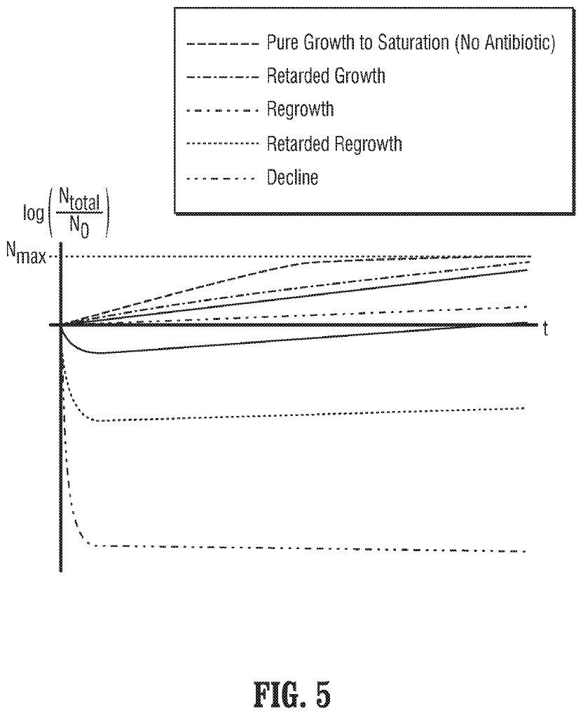

Indeed, when a bacterial population is in decline (in response to antibiotic exposure) optical density measurements produce a continuous non-decreasing signal, because the sum of live and dead cells is non-decreasing FIG. 5. In particular, optical density measurements would be of little value in the important case of time kill experiments with bacterial populations comprising subpopulations of varying degrees of antibiotic resistance, because, at certain concentrations of the antibiotic, regrowth of the population would occur, due to early decline of susceptible subpopulations and late growth of subpopulations resistant to the antibiotic, as shown in FIG. 5. In that figure, the thick curves corresponding to both live and dead cell counts of a growing bacterial population (at low antibiotic concentrations) provide enough qualitative information on live cell counts (thin lines) by inspection. However, for populations in regrowth, in retarded regrowth, or in decline (FIG. 5), mere inspection of the thick lines offers no indication about the trend of live cell counts (thin lines) and offers hardly any clues towards the design of an effective treatment. It is for these situations, which are essential from a therapeutic viewpoint, that the mathematical model-based method disclosed herein provides a general solution.

The approach taken to build this mathematical model structure starts with equations that capture the effect of an antibiotic on a heterogeneous bacterial population comprising subpopulations of varying degrees of resistance, as shown qualitatively in FIG. 4 (Bhagunde P R et al., (2015) Aiche Journal 61 (8):2385-2393; Nikolaou M and Tam V H (2006) Journal of Mathematical Biology 52 (2):154-182). That model structure is extended to describe the effect of an antibiotic on the entire cell count in a bacterial population, including both live and dead cells, as illustrated in FIG. 5. As detailed below, the disclosed model structure relies on minimal assumptions and includes a small number of parameters that can be easily estimated based on experimental data.

Provided herein are the basic equations that constitute the starting point for the main results, which are developed in the Mathematical Modeling section and illustrated through an experimental study presented below.

### Materials and Methods

Background on Mathematical Modeling. When a bacterial population is exposed to an antibiotic, the population experiences kill rates r≥0 (Giraldo J et al., (2002) Pharmacology & Therapeutics 95:21-45; Justco et al., (1971) J Pharm Sci 60:892-895; Wagner J (1968) J Theoret Biol 20:173-201) that vary over subpopulations of the overall population, as these subpopulations have different susceptibilities to the antibiotic at a set concentration (Giraldo J. et al. (2002) Pharmacology & Therapeutics 95:21-45; Lipsitch M, et al., (1997) Antimicrobial Agents and Chemotherapy 41 (2):363-373). If such a heterogeneous bacterial population is exposed to an antibiotic at time-invariant concentration, the distribution of kill rates changes over time, as susceptible bacteria are killed faster than less susceptible (more resistant) bacteria, thus changing the pharmacodynamics of the antibiotic/bacteria interaction. The least susceptible (most resistant) subpopulation eventually becomes dominant and experiences either eradication or regrowth, depending on whether the natural growth rate of that most resistant subpopulation is lower or higher, respectively, than the kill rate induced on that subpopulation by the antibiotic at that concentration (Giraldo J. et al. (2002) Pharmacology & Therapeutics 95:21-45; 27-29; Jusko W (1971) J Pharm Sci 60:892-895; Wagner J (1968) J Theoret Biol 20:173-201; Hill A V (1910) J Physiol 40:iv-vii).

It can be shown (Nikolaou M, Tam V H (2006) Journal of Mathematical Biology 52 (2):154-182. doi:10.1007/s00285-005-0350-6; Mytilinaios I S, et al., (2012) International Journal of Food Microbiology 154 (3):169-176) that under realistic assumptions the size of a heterogeneous bacterial population exposed to an antibiotic at constant concentration over time is well approximated by the equation

$$ln [ \frac{{N}_{live} ( t )}{{N}_{0}} ] = ( {K}_{g} - {r}_{m i n} ) t + λ ( {e}^{- at} - 1 ) - ln [ 1 + {K}_{g} \frac{{N}_{0}}{{N}_{m a x}} {\int }_{0}^{t} exp [ ( {K}_{g} - {r}_{m i n} ) τ + λ ( {e}^{- a τ} - 1 ) ] d τ ] \qquad ( 3 )$$

the average kill rate over time is well approximated by the equation

$$μ ( t ) = {r}_{m i n} + ( μ - {r}_{m i n} ) exp [ - \frac{μ - {r}_{m i n}}{λ} t ] = {r}_{m i n} + λ {ae}^{- at} \qquad ( 4 )$$

and the variance of the kill rate over time is well approximated by

$${σ ( t )}^{2} = \frac{{( μ - {r}_{m i n} )}^{2}}{λ} exp [ - \frac{μ - {r}_{m i n}}{λ} t ] = λ {a}^{2} {e}^{- at} \qquad ( 5 )$$

where
Nₗᵢᵥₑ(t) is the live bacterial population size with initial value N₀
K_{g} is the physiological net growth rate of the entire bacterial population, common for all subpopulations
rₘᵢₙ is the kill rate induced by the antibiotic on the most resistant (least susceptible) subpopulation
Nₘₐₓ is the maximum size of a bacterial population reaching saturation under growth conditions
μ(t) is the kill rate average over the bacterial population at time t
σ(t)² is the kill rate variance over the bacterial population at time t
λ>0, a>0 are constants associated with the initial decline of the average kill rate of the population, and correspond to the Poisson distributed variable

$$\frac{r - {r}_{min}}{a}$$

with average and variance equal to λ.

Note that the above two equations for Nₗᵢᵥₑ(t) and μ(t) have been derived with essentially no assumptions about the mechanisms that may confer bacterial resistance. The parameters K_{g}, rₘᵢₙ, λ, a, Nₘₐₓ that appear in the above equations can be estimated from time kill experiments that produce measurements of Nₗᵢᵥₑ (t) over time at various set concentrations of the antibiotic.

Estimates of parameters such as K_{g} and rₘᵢₙ are essential for guiding the design of effective dosing regimens. For example, it has been shown by Nikolaou M, et al. (Ann Biomed Eng 35 (8):1458-1470) that an antibiotic injected periodically and following pharmacokinetics of exponential decay during each period, T, is effective against a heterogeneous bacterial population when

$$\frac{1}{T} {\int }_{0}^{T} {r}_{min} ( C ( t ) ) d t > {K}_{g} \qquad ( 6 )$$

where rₘᵢₙ(C(t)) is the kill rate of the most resistant subpopulation as a function of antibiotic concentration C(t), typically an expression of the type (Giraldo J et al., (2002) Pharmacology & Therapeutics 95:21-45; Jusko W (1971) J Pharm Sci 60:892-89; Wagner J (1968); J Theoret Biol 20:173-201).

$${r}_{min} ( C ) = {K}_{k} \frac{{C}^{H}}{{C}^{H} + {C}_{50}^{H}} \qquad ( 7 )$$

where Kₖ is the maximal kill rate achieved as C→∞; C₅₀ is a constant equal to the antimicrobial agent concentration at which 50% of the maximal kill rate is achieved; and H is the Hill exponent (Hill A V (1910) J Physiol 40:iv-vii), corresponding to how inflected r is as a function of C.

To estimate model parameters, measurements of Nₗᵢᵥₑ (t) can be typically obtained by drawing small samples from the bacterial population at distinct points in time and using standard plating methods (Sanders E R (2012) Journal of Visualized Experiments (63):e3064). While this measurement approach is well established, it is laborious, time-consuming, and can produce measurements at only a few distinct points in time under realistic conditions. By contrast, simple optical methods produce an essentially continuous signal for the bacterial population size over time. The Achilles heel of these methods, as already mentioned, is that while they produce a signal for a bacterial population in growth that is easy to interpret by inspection, when the bacterial population of live cells is in decline (because of antibiotic exposure) the optical density signal produced is practically impossible to interpret by inspection. Therefore, there is an incentive to develop equations that capture the pharmacodynamics of the combined population of both live and dead cells exposed to an antibiotic, as counterparts of eqns. (3) and (5). The utility of such equations would be in inferring profiles over time for live cell counts from measurements of total (both live and dead) cell counts in a population. That information would guide decisions about effective use of antibiotics, particularly to eradicate bacterial populations that exhibit varying degrees of resistance to one or multiple antibiotics. The development of such equations is discussed in the next section.

Antibiotic Agent. Levofloxacin (LVX) powder was a gift from Achaogen (South San Francisco, Calif.). A stock solution at 1024 μg/mL in sterile water has been prepared ahead of time and stored in −70° C. For each experimental study, the drug was diluted to the optimum concentration through standard lab techniques.

Microorganism. A laboratory reference wild type Acinetobacter baumannii (AB), ATCC BAA747, was utilized in the study. The bacteria were stored at −70° C. in Protect® vials. Before the experiment, the bacteria were subcultured at least twice on 5% blood agar plates for 24 hours at 35° C. and fresh colonies were used. The susceptibility (MIC) to LVX was previously found to be 0.25 μg/mL.

Optical density measurements. Real time measurements of the bacterial population size are provided by an optical instrument (model 216Dx), provided by BacterioScan® (St. Louis, Mo.). The instrument uses laser light scattering coupled with traditional optical density measurements to provide a quantitative measure of particle (e.g. bacterial) density in liquid samples. Prepared samples were loaded into custom, sterilized cartridges and inserted into instruments for automated optical profiling. The instrument utilizes a 650 nm wavelength laser that is passed through the liquid sample (with a 2.5 cm pathlength) and collects both the scattered light as well as the unscattered light (no particle interaction) signals. Using a proprietary algorithm, these signals are converted to numeric values and scaled to bacterial colony forming units per milliliter (CFU/ml) based on average size and density standards for typical bacterial cells. In its current version, the instrument allows for simultaneous measurements of 16 individual combinations of antibiotic and bacterial population in suspension maintained at 35° C. Full computer connectivity allows continuous monitoring, storage, and transfer of all measurements.

Bacterial susceptibility studies. Bacteria were initially grown in a temperature regulated shaker bath to log phase growth and diluted to a concentration of

$$1 {0}^{5} - 1 {0}^{5.5} \frac{CFU}{mL} .$$

The initial target concentration was estimated by absorbance values at 630 nm. Samples of the bacterial population at the desired initial concentration were transferred to six temperature regulated flasks with cation adjusted Mueller Hinton broth and LVX concentrations of {0, 0.5, 2, 8, 16, 32}×MIC. Serial samples were taken in duplicate from each flask at 0, 2, 4, 8, and 24 hours. Each sample containing antibiotic was first centrifuged to remove the supernatant antibiotic solution, replace it with an equal volume of sterile saline to minimize drug carryover effect, and was subsequently plated quantitatively to determine viable bacterial burden. The preceding procedure was repeated three times on different days.

Optical instrument susceptibility studies. Bacteria were initially grown in a temperature regulated shaker bath to log phase growth and diluted to a concentration of

$${10}^{5} - {10}^{5.5} \frac{CFU}{mL} .$$

The initial target concentration was estimated by absorbance values at 630 nm. Samples of the bacterial population at the desired initial concentration were transferred to 4 temperature regulated covets inside the optical instrument with cation adjusted Mueller Hinton broth and LVX concentrations of {0, 0.5, 2, 8}×MIC. The instrument took serial samples automatically from each flask approximately every 1 minute for 48 hours. The preceding procedure was repeated three times on different days.

Data fit. Eqn. (3) was used to fit data from the viability plating experiments described above. Similarly eqns. (19) or (2) were used to fit data from the optical density instrument. Parameter estimates were provided by MS Excel® and Mathematica®. Because estimates of K_{d} could not be obtained directly from plating data, they were obtained from fitting eqns. (19) or (2) to data produced by the optical density instrument, with all remaining parameters set at their values estimated from plating data.

### Results

Mathematical modeling. One proceeds to the step-by-step derivation of the dynamics and analytical expression for the entire size of a heterogeneous bacterial population exposed to an antibiotic at a time-invariant concentration. This is a typical setting in time kill experiments, as the data it produces, particularly if successfully modeled, can be well used to analyze the effect of antibiotics at time-varying concentrations corresponding to realistic pharmacokinetics of clinical significance Nikolaou M, Schilling A N, Vo G, Chang K T, Tam V H (2007) Modeling of microbial population responses to time-periodic concentrations of antimicrobial agents. (Ann Biomed Eng 35 (8):1458-1470).

It can be shown (Bhagunde P R et al., (2015) Aiche Journal 61 (8):2385-2393) that when a heterogeneous bacterial population is exposed to an antibiotic, the dynamics of the population of live bacterial cells becomes

$$\frac{{dN}_{live}}{dt} = {\underbrace{{K}_{g} [ 1 - \frac{{N}_{live} ( t )}{{N}_{m a x}} ] {N}_{live} ( t )}}_{Net physiological growth} - {\underbrace{μ ( t ) {N}_{live} ( t )}}_{\begin{matrix}Kill rate induced \\ by antibiotic\end{matrix}} \qquad ( 8 )$$

Similarly, the dynamics of the population of dead cells becomes

$$\frac{{dN}_{dead}}{dt} = {\underbrace{{K}_{d} {N}_{live} ( t )}}_{Physiological death} + {\underbrace{μ ( t ) {N}_{live} ( t )}}_{\begin{matrix}Killing induced \\ by antibiotic\end{matrix}} = ( {K}_{d} + μ ( t ) ) {N}_{live} ( t ) \qquad ( 9 )$$

Adding the above two equations yields the following equation for the entire population

Nₜₒₜₐₗ=Nₗᵢᵥₑ+N_{dead} (10)

of live and dead cells:

$$\frac{{dN}_{total}}{dt} = \frac{d ( {N}_{live} + {N}_{dead} )}{dt} = {\underbrace{{K}_{g} [ 1 - \frac{{N}_{live} ( t )}{{N}_{m a x}} ] {N}_{live} ( t )}}_{Net physiological growth} + {\underbrace{{K}_{d} {N}_{live} ( t )}}_{Death} = ( {K}_{d} + {K}_{g} [ 1 - \frac{{N}_{live} ( t )}{{N}_{m a x}} ] ) {N}_{live} ( t ) = ( {K}_{b} - {K}_{g} \frac{{N}_{live} ( t )}{{N}_{m a x}} ) {N}_{live} ( t ) \qquad ( 11 )$$

where the connection between the constants K_{g}, K_{b}, and K_{b} is discussed in Appendix B.

Combination of the above equations immediately implies

$$\frac{{dN}_{live}}{dt} = ( - {K}_{g} \frac{{N}_{live} ( t )}{{N}_{m a x}} + {K}_{g} - {r}_{m i n} - λ {ae}^{- at} ) {N}_{live} ( t ) \qquad ( 12 )$$

$$\frac{{dN}_{total}}{dt} = ( {K}_{g} [ 1 - \frac{{N}_{live} ( t )}{{N}_{m a x}} ] + {K}_{d} ) {N}_{live} ( t ) = ( - {K}_{g} \frac{{N}_{live} ( t )}{{N}_{m a x}} + {K}_{b} ) {N}_{live} ( t ) \qquad ( 13 )$$

Note that from the above eqn. (12) it is clear that the bacterial population can be eventually eradicated if and only if

rₘᵢₙ>K_{g} (14)

The above eqns (12) and (13) can be solved analytically to provide closed form expressions for Nₜₒₜₐₗ(t), as discussed below.
Nₜₒₜₐₗ(t) for growing population (no antibiotic)
In the absence of antibiotic, eqn. (12) yields

$$\frac{{dN}_{live}}{dt} = {K}_{g} [ 1 - \frac{{N}_{live} ( t )}{{N}_{m a x}} ] {N}_{live} ( t )$$

which yields

$${N}_{live} ( t ) = {N}_{0} \frac{1}{\frac{{N}_{0}}{{N}_{m a x}} + {e}^{- {K}_{g} t} ( 1 - \frac{{N}_{0}}{{N}_{m a x}} )} \qquad ( 15 )$$

Substituting the above Nₗᵢᵥₑ(t) into eqn. (13) and integrating yields

$$\frac{{N}_{total} ( t )}{{N}_{0}} = \frac{1}{\frac{{N}_{0}}{{N}_{max}} + {e}^{- {K}_{g}^{t} ( 1 - \frac{{N}_{0}}{{N}_{max}} )}} + \frac{{N}_{max}}{{N}_{0}} \frac{{K}_{d}}{{K}_{g}} ln [ ( {e}^{{K}_{g}^{t}} - 1 ) \frac{{N}_{0}}{{N}_{max}} + 1 ] \qquad ( 16 )$$

Note the asymptotic behavior of the above eqn. (16):
For t≈0 with an initial bacterial population size well below its saturation point,

$$\frac{{N}_{0}}{{N}_{m a x}} \approx 0 which implies \\ \frac{{N}_{total} ( t )}{{N}_{0}} \approx ( ( 1 + \frac{{K}_{d}}{{K}_{g}} ) {e}^{{K}_{g} t} - \frac{{K}_{d}}{{K}_{g}} ) \qquad ( 17 )$$

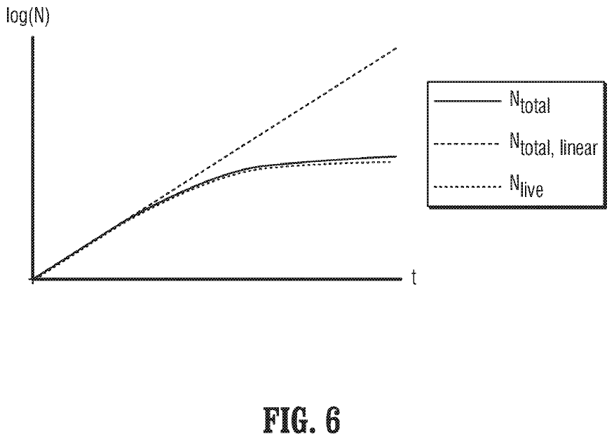

Typical profiles for each of eqns. (15), (16), and (17) are shown FIG. 6.
   For t→∞ one gets

$$\frac{{N}_{total} ( t )}{{N}_{0}} \approx \frac{{N}_{m a x}}{{N}_{0}} {K}_{d} t \qquad ( 18 )$$

### General Population Exposed to Antibiotic.

In the presence of an antibiotic, eqn. (12) eventually yields (See, APPENDIX C)

$$\frac{{N}_{total} ( t )}{{N}_{0}} = \frac{{e}^{λ ( {e}^{- at} - 1 ) + ( {K}_{g} - {r}_{min} ) t}}{1 + {K}_{g} \frac{{N}_{0} {e}^{- λ}}{{N}_{max} a} {λ}^{\frac{{K}_{g} - {r}_{min}}{a}} {\int }_{λ {e}^{- a t}}^{λ} {z}^{- 1 + \frac{{r}_{min} - {K}_{g}}{a}} {e}^{z} dz} ++ {\int }_{0}^{t} \frac{( {K}_{d} + {r}_{min} + λ {ae}^{- a τ} ) {e}^{λ ( {e}^{- a τ} - 1 ) + ( {K}_{g} - {r}_{min} ) τ}}{1 + {K}_{g} \frac{{N}_{0} {e}^{- λ}}{{N}_{max} a} {λ}^{\frac{{K}_{g} - {r}_{min}}{a}} {\int }_{λ {e}^{- a τ}}^{λ} {z}^{- 1 + \frac{{r}_{min} - {K}_{g}}{a}} {e}^{z} dz} d τ \qquad ( 19 )$$

Note that when the initial population is far from its saturation point, i.e.

$$\frac{{N}_{0}}{{N}_{max}} \approx 0 ,$$

eqn. (12) yields

$$\frac{{N}_{live} ( t )}{{N}_{0}} = {e}^{λ ( {e}^{- a t} - 1 ) + ( {K}_{g} - {r}_{min} ) t} \qquad ( 20 )$$

which implies that eqn. (19) can be simplified as

$$\frac{{N}_{total} ( t )}{{N}_{0}} = {e}^{λ ( {e}^{- a t} - 1 ) + ( {K}_{g} - {r}_{min} ) t} ++ {e}^{- λ} {λ}^{\frac{{K}_{g} - {r}_{min}}{a}} ( \frac{{K}_{d} + {r}_{min}}{a} {\int }_{λ {e}^{- a t}}^{λ} {z}^{- 1 + \frac{{r}_{min} - {K}_{g}}{a}} {e}^{z} dz + {\int }_{λ {e}^{- a t}}^{λ} {z}^{\frac{{r}_{min} - {K}_{g}}{a}} {e}^{z} dz ) \qquad ( 2 )$$

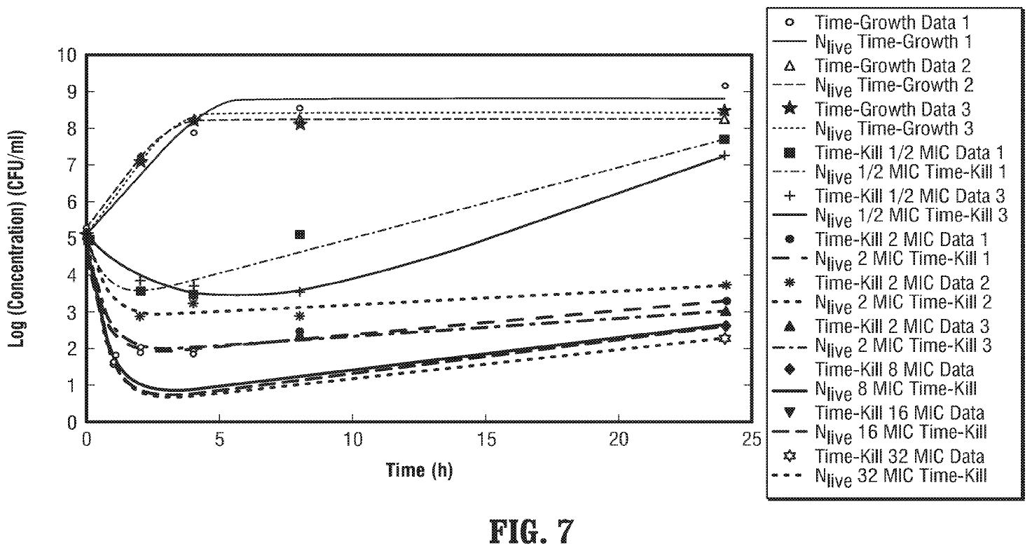

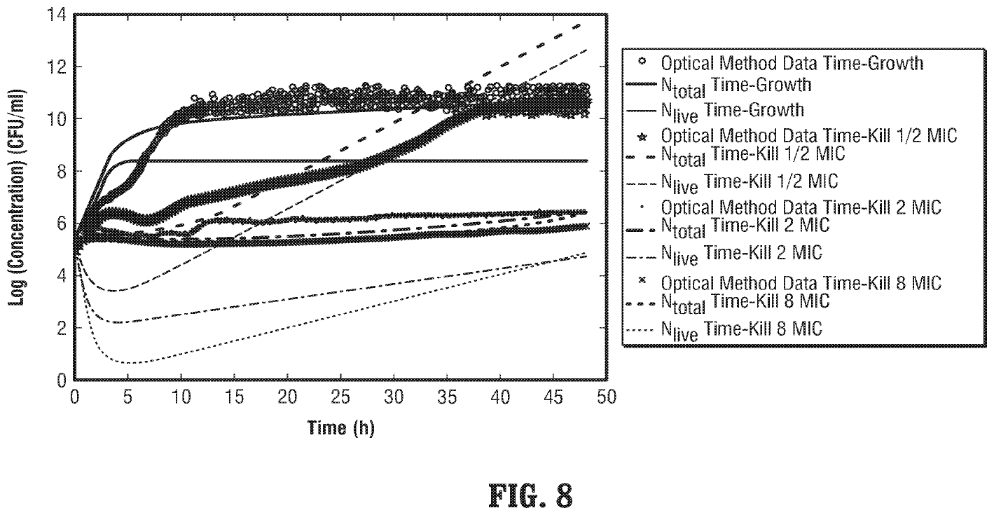

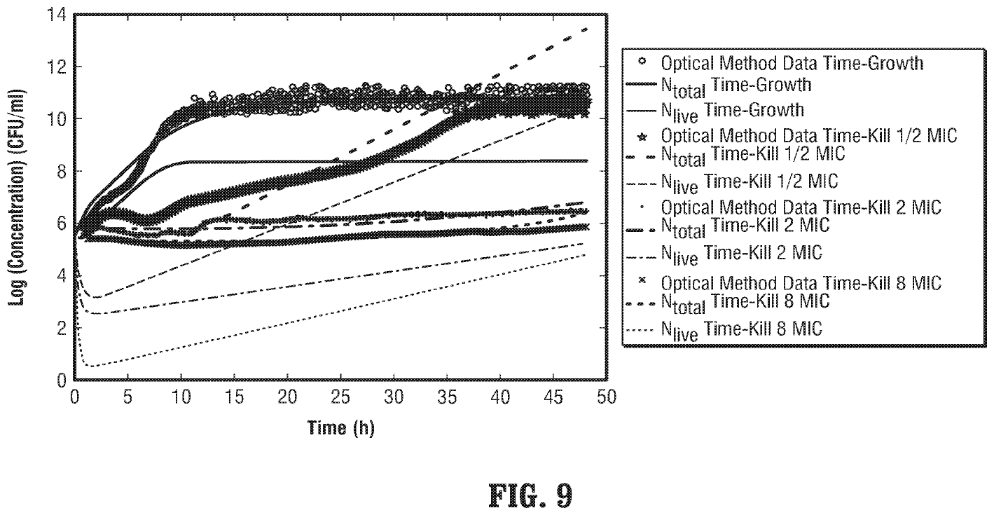

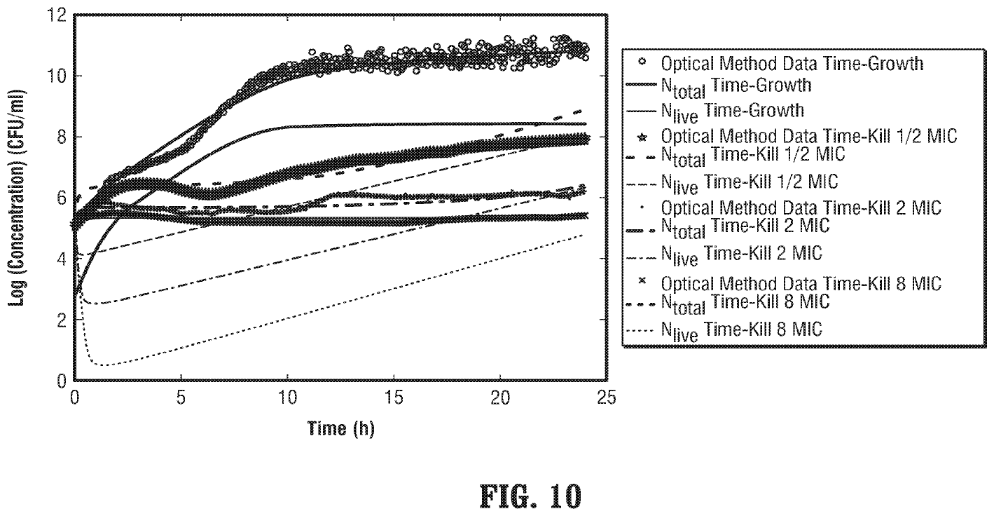

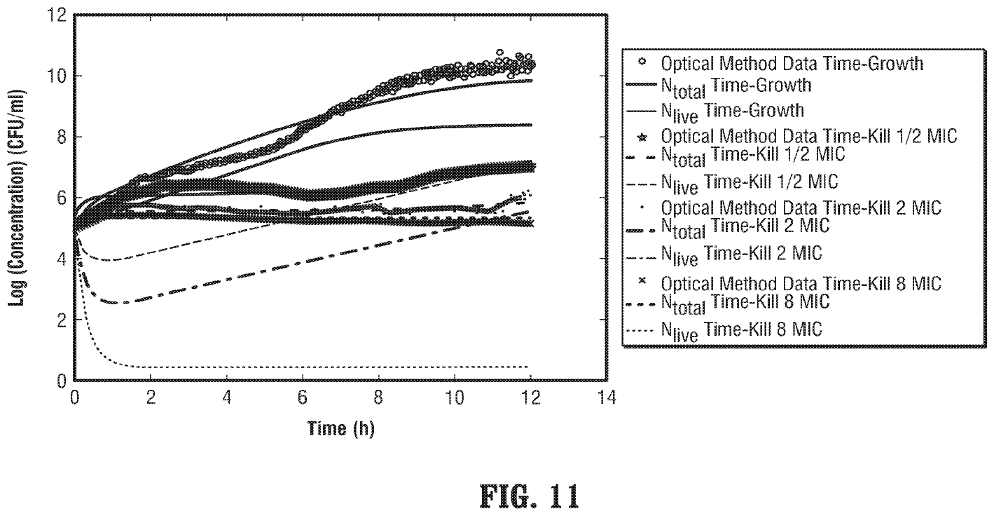

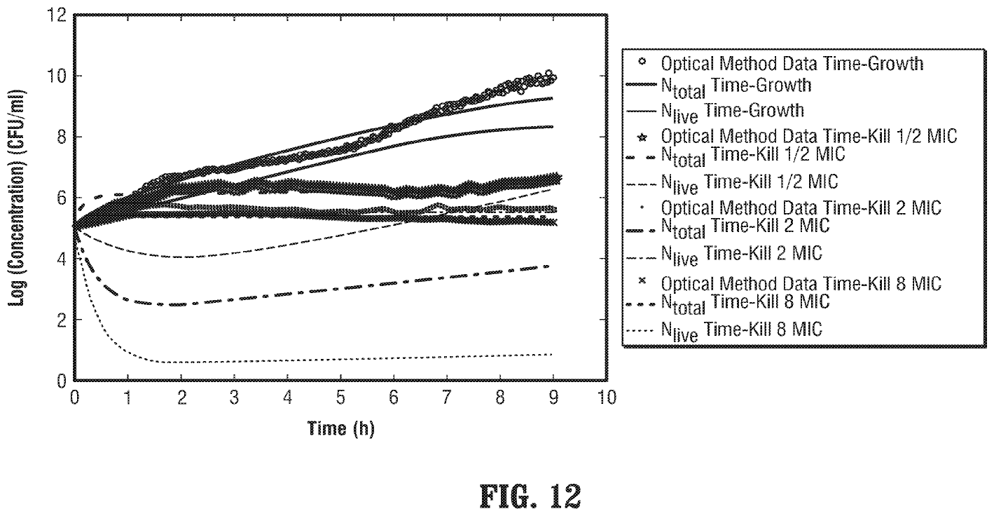

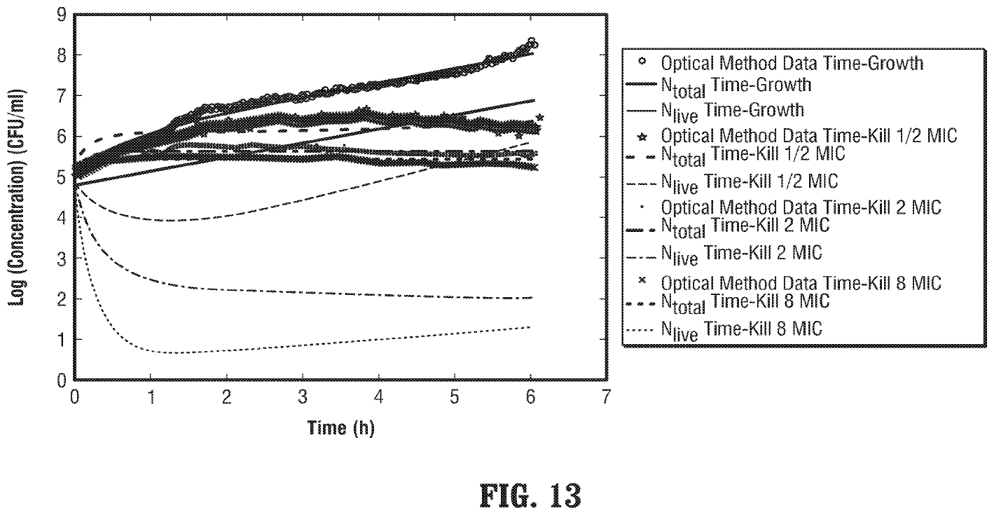

To illustrate the applicability of the mathematical modeling framework presented above, the following results were obtained. These results were used to compare live bacteria counts estimated by plating to counts estimated by optical density measurements and the proposed model-based method:
a. Use of eqn. (1) to fit experimental data on Nₗᵢᵥₑ produced using standard viability plating methods, as mentioned above (FIG. 7). Parameter estimates are shown in Table 1.
b. Use of the parameter estimates of part (a.) into eqn. (2), to produce values of Nₜₒₜₐₗ(t) and compare these values to data produced experimentally using optical density measurements (FIG. 8). Values of Nₗᵢᵥₑ(t) using the above parameter estimates into eqn. Eq. were also produced, for comparison to FIG. 7.
c. Use of eqn (2) to fit data on Nₜₒₜₐₗ produced experimentally using optical density measurements (FIG. 9). Parameter estimates are shown in Table 1.
d. Use of eqn. (2) to fit data on Nₜₒₜₐₗ was repeated for data produced experimentally over a period of 24 hours (FIG. 10), 12 hours (FIG. 11), 9 hours (FIG. 12), and 6 hours (FIG. 13). Corresponding parameter estimates are shown in Table 1.

TABLE 1
Parameter estimates for models in eqns. (1) and (2)⁺

| Plating | Plating | Plating | Instrument | Instrument | Instrument | Instrument | Instrument |
|---|---|---|---|---|---|---|---|
| 1 | 2 | 3 | 48 h* | 24 h* | 12 h* | 9 h* | 6 h* |

Time Growth Placebo

| | | | | | | | | |
|---|---|---|---|---|---|---|---|---|
| log Nₘₐₓ | 8.8 ± 0.1 | 8.2 ± 0.01 | 8.4 ± 0.05 | 8.4 ± 0.03 | 8.4 ± 0.05 | 8.4 ± 0.07 | 8.4 ± 0.14 | 8.4 ± 6 |
| K_{g} | 1.8 ± 0.1 | 2.2 ± 0.03 | 2.3 ± 0.09 | 0.9 ± 0.02 | 1 ± 0.03 | 1 ± 0.03 | 1 ± 0.03 | 0.8 ± 0.1 |
| K_{d} | 4.0** | 4.0** | 4.0** | 10 ± 0.75 | 17 ± 2 | 6.3 ± 0.7 | 3.7 ± 0.3 | 10.7 ± 1.4 |

Time Kill ½ MIC

| | | | | | | | | |
|---|---|---|---|---|---|---|---|---|
| μ₀ | 8.1 ± 0.3 | 12 ± 0.4 | 4.0 ± 0.7 | 10 ± 0.4 | 12 ± 0.16 | 11 ± 0.03 | 4.2 ± 0.04 | 5 ± 0.2 |
| σ₀ | 3.1 ± 0.1 | 4.5 ± 0.1 | 0.74 ± 0.36 | 4.1 ± 0.14 | 10.8 ± 0.1 | 6 ± 0.01 | 1.8 ± 0.02 | 2.5 ± 0.1 |
| α | 1.4 ± 0.1 | 2.1 ± 0.1 | 0.24 ± 0.9 | 1.8 ± 0.24 | 10.4 ± 0.16 | 3.3 ± 0.01 | 0.76 ± 0.02 | 1.2 ± 0.1 |

Time Kill 2 MIC

| | | | | | | | | |
|---|---|---|---|---|---|---|---|---|
| μ₀ | 15 ± 0.6 | 11 ± 0.38 | 13 ± 0.53 | 16 ± 0.1 | 22 ± 0.06 | 21 ± 0.03 | 15 ± 0.1 | 15.5 ± 0.3 |
| σ₀ | 4.6 ± 0.1 | 3.6 ± 0.10 | 4.0 ± 0.12 | 6.1 ± 0.03 | 9.6 ± 0.01 | 8.4 ± 0.01 | 5.5 ± 0.02 | 6.1 ± 0.15 |
| α | 1.6 ± 0.1 | 1.5 ± 0.11 | 1.5 ± 0.12 | 2.5 ± 0.04 | 4.3 ± 0.01 | 3.2 ± 0.01 | 2.1 ± 0.2 | 2.6 ± 0.3 |

Time Kill 8 MIC

| | | | | | | |
|---|---|---|---|---|---|---|
| μ₀ | 16 ± 0.62 | 34 ± 0.05 | 28 ± 0.2 | 35 ± 0.1 | 26 ± 0.06 | 33 ± 0.3 |
| σ₀ | 4.4 ± 0.12 | 9.9 ± 0.01 | 8.6 ± 0.04 | 10.5 ± 0.02 | 7.7 ± 0.02 | 10.4 ± 0.1 |
| α | 1.3 ± 0.10 | 3.0 ± 0.01 | 2.7 ± 0.03 | 3.2 ± 0.06 | 2.4 ± 0.04 | 3.3 ± 0.2 |

⁺Parameters for plating experiments conducted at 16 and 32 MIC not reported, as corresponding experiments were not conducted with the optical density instrument at these concentrations.
*Standard errors should be interpreted with caution, as small systematic errors are also present
**Please see discussion below about this estimate.

The results in FIG. 7 through FIG. 13 and Table 1 demonstrate that the mathematical framework developed in the mathematical modeling section makes it feasible to estimate the number of live cells, Nₗᵢᵥₑ(t), from optical measurements of the entire number of both live and dead cells, Nₜₒₜₐₗ(t), over time. This fundamental capability provides the ability for optical density measurements to be routinely used as a highly efficient tool for discerning the pharmacodynamics of bacteria/antibiotic interaction and use the outcome towards the design of personalized therapeutic treatments.

More specifically, the analytical expression derived for the time-dependent size of a heterogeneous bacterial population (eqn. (2)) was shown to be the key for analyzing optical data. Indeed, the curves for Nₜₒₜₐₗ produced by eqn. (2) using parameter estimates from fitting Nₗᵢᵥₑ to plating data agree well with Nₜₒₜₐₗ from the optical density measurements (FIG. 8). Furthermore, fitting eqn. (2) to the optical density data produces curves for Nₗᵢᵥₑ in FIG. 9 that are reasonably close to those of FIG. 8. More importantly, the estimates of Nₗᵢᵥₑ produced from fitting the model to optical density measurements alone are close to estimates from direct measurement of Nₗᵢᵥₑ produced through plating. Finally, estimates of Nₗᵢᵥₑ produced by fitting experimental data over shorter periods of time 24, 12, 9, and 6 hours in FIG. 10 through FIG. 13, respectively) demonstrated the robustness of the method, in that Nₗᵢᵥₑ estimates remained close to one another in all cases. It should be stressed that getting reasonable estimates over short time periods is of paramount importance for use of the approach to rapidly design therapeutic treatments.

In all optical density measurements collected (FIG. 8 through FIG. 13), small systematic errors are evident. For example, the optical measurement curves in FIG. 8 through FIG. 13 exhibit a temporary reduction in growth rate starting at around 4 hours. The growth rate resumes its previous value at around 6 hours. Finally, it starts to plateau at around 10 hours. The growth rate fluctuation from an initial value to a lower one and back is purely an artifact of the instrument used, as different optical methods (diffraction and absorption) are used in different time regimes for cell counting. The time growth curve is essential for the reproduction of the time kill curves as it provides the natural death rate parameter of the bacteria, K_{d}. Thus, small fluctuations of the time growth model fit to the total number of bacteria has great impact to the actual living bacteria present as well as to the time kill results. Artificial fluctuations are also noticed in the time kill curves at around 6 hours and 24-35 hours for ½×MIC, at 12 hours for the 2×MIC curve and slightly later for the 8×MIC curve.

As expected, the model fits to data collected over different time periods will be impacted by these fluctuations. Indeed, proceeding from FIG. 9 (fit of optical density measurements over 48 hours) FIG. 10 (fit of optical density measurements over 24 hours) there is no significant deviation, except for the ½×MIC case, but even in this case the Nₗᵢᵥₑ curves produced by the model are reasonably close to each other. Continuing to FIG. 11 (fit of optical density measurements over 12 hours), the Nₗᵢᵥₑ curve at 8×MIC produced by the model plateaus completely after 1 hour, a deviation from corresponding curves in FIG. 9 and FIG. 10. This is because the optical instrument used has not shifted measurement mode in the time allotted (12 hours) and the population exhibits a slightly decreasing curve. The analysis shows that such a curve represents a complete eradication of bacteria by the antibiotic at the corresponding concentration of 8×MIC. A similar phenomenon can be observed in FIG. 12 and in FIG. 13 where 2×MIC starts to show a greater inhibition to bacteria and later complete eradication of the bacterial population accordingly.

The information contained in the fitted model could be used in the design of effective therapies against challenging infections, e.g. by ensuring that rₘᵢₙ>K₉ or by ensuring that eqn. (6) is satisfied. This underscores the important role of using the proposed mathematical modeling framework to extract information on a declining population from measurements that could not possibly detect it, and to use such information effectively. A mathematical model-based method was developed to glean in vitro pharmacodynamics from otherwise unusable optical density measurements collected in time kill experiments of bacterial populations exposed to antibiotics. The model-based method was applied to experimental optical density measurements over time, and produced estimates of live bacteria counts in agreement with counts produced manually by a standard plating method at a few sampling points. The mathematical model-based method disclosed herein helps retain all of the advantages associated with optical density measurements, while removing their basic disadvantage, namely their inability to distinguish between live and dead cells and thus track the size of a bacterial population in decline from exposure to antibiotics. This model-based method permits rapid systematic design of effective personalized dosing regimens against resistant bacteria. As development of optical density measurement technology progresses further, e.g. by simplifying calibration or by extending the dynamic range (Pla M L, Oltra S, Esteban M D, Andreu S, Palop A (2015) BioMed Research International 2015:14; Mytilinaios I S et al., (2012) International Journal of Food Microbiology 154 (3):169-176; López S, et al., (2004) International Journal of Food Microbiology 96 (3):289-300), it is anticipated that use of the model-based method presented here will prove useful at improving therapeutic outcomes in treatment of resistant clinical infections.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that this disclosure is not limited to the precise embodiments described, and that various other changes and modifications may be effected by one skilled in the art without departing from the scope or spirit of this disclosure. Additionally, the elements and features shown or described in connection with certain embodiments may be combined with the elements and features of certain other embodiments without departing from the scope of this disclosure, and that such modifications and variations are also included within the scope of this disclosure. Accordingly, the subject matter of this disclosure is not limited by what has been particularly shown and described.

### APPENDIX A

### Develop Basic Equations

### Population of Live Cells

As shown in previous work (Bhagunde et al., AlChE J., in print, 2015):

$$\frac{d {N}_{live}}{dt} = ( {K}_{g} [ 1 - \frac{{N}_{live} ( t )}{{N}_{max}} ] - μ ( t ) ) {N}_{live} ( t ) \qquad ( 1 )$$

$$ln [ \frac{{N}_{live} ( t )}{{N}_{0}} ] = ( {K}_{g} - {r}_{min} ) t + ( {e}^{- at} - 1 ) - ln [ 1 + {K}_{g} \frac{{N}_{0}}{{N}_{max}} {\int }_{0}^{t} exp [ ( {K}_{g} - {r}_{min} ) τ + λ ( {e}^{- a τ} - 1 ) ] d τ ] \qquad ( 2 )$$

$$μ ( t ) = {r}_{min} + ( μ - {r}_{min} ) exp [ - \frac{μ - {r}_{min}}{λ} t ] = {r}_{min} + λ {ae}^{- at} \\ and \qquad ( 3 )$$

$${σ ( t )}^{2} = \frac{{( μ - {r}_{min} )}^{2}}{λ} exp [ - \frac{μ - {r}_{min}}{λ} t ] = λ {a}^{2} {e}^{- at} \qquad ( 4 )$$

respectively, where

$${\int }_{0}^{t} exp [ ( {K}_{g} - {r}_{min} ) τ + λ ( {e}^{- a τ} - 1 ) ] d τ = \frac{{e}^{- λ}}{a} {λ}^{\frac{{K}_{g} - {r}_{min}}{a}} {\int }_{λ {e}^{- at}}^{λ z} {z}^{\frac{{r}_{min} - {k}_{g}}{a} - 1} {e}^{z} dz \qquad ( 5 )$$

and
the parameters rₘᵢₙ, a, μ, and

$$λ = \frac{μ - {r}_{min}}{a}$$

depend on the antibiotic concentration C;
K_{g}=K_{b}−K_{d} is the net physiological growth of bacteria, equal to the difference between the physiological birth and death rates, K_{b} and K_{d}, respectively;
μ(t) is the average antibiotic-induced rate of bacteria; and
the incomplete gamma function (related to some of the above integrals) is defined as

$$Γ ( c , {z}_{0} , {z}_{1} ) {=}^{} {\int }_{{z}_{0}}^{{z}_{2}} {z}^{c - 1} {e}^{- z} dz . \qquad ( 6 )$$

Connection Between K_{g}, K_{b}, and K_{d}
In the absence of an antibiotic, Eqn. (1) implies

$$\frac{d {N}_{live}}{dt} = {\underbrace{{K}_{b} [ 1 - \frac{{N}_{live} ( t )}{{N}_{c}} ] {N}_{live} ( t )}}_{Physiological birth} - {\underbrace{{K}_{d} {N}_{live} ( t )}}_{Physiological death} = {\underbrace{( {K}_{b} - {K}_{d} )}}_{{K}_{g}} [ 1 - \frac{{K}_{b}}{{K}_{b} - {K}_{d}} \frac{{N}_{live} ( t )}{{N}_{c}} ] {N}_{live} ( t ) = {\underbrace{{K}_{g} [ 1 - \frac{{N}_{live} ( t )}{{N}_{max}} ] {N}_{live} ( t )}}_{Net physiological growth} with \qquad ( 7 )$$

$${N}_{max} = {N}_{c} \frac{{K}_{b} - {K}_{d}}{{K}_{b}} = {N}_{c} \frac{{K}_{g}}{{K}_{b}} \\ and \qquad ( 8 )$$

$${K}_{g} = {K}_{b} - {K}_{d} \qquad ( 9 )$$

### Populations of Dead and Live Cells

in the presence of an antibiotic, eqn. (1) implies

$$\frac{d {N}_{live}}{dt} = {\underbrace{{K}_{g} [ 1 - \frac{{N}_{live} ( t )}{{N}_{max}} ] {N}_{live} ( t )}}_{Net Physiological growth} - {\underbrace{μ ( t ) {N}_{live} ( t )}}_{\begin{matrix}Kill rate induced \\ by antibiotic\end{matrix}} \\ with \qquad ( 10 )$$

$$μ ( t ) = {r}_{min} + λ {ae}^{- at} \\ and \qquad ( 11 )$$

$$\frac{d {N}_{dead}}{dt} = {\underbrace{{K}_{d} {N}_{live} ( t )}}_{\begin{matrix}Physiological \\ death\end{matrix}} + {\underbrace{μ ( t ) {N}_{live} ( t )}}_{\begin{matrix}Killing induced \\ by antibiotic\end{matrix}} = ( {K}_{d} + μ ( t ) ) {N}_{live} ( t ) \\ \qquad ( 12 )$$

$$\frac{{dN}_{total}}{dt} = \frac{d ( {N}_{live} + {N}_{deed} )}{dt} = {\underbrace{{K}_{g} [ 1 - \frac{{N}_{live} ( t )}{{N}_{max}} ] {N}_{live} ( t )}}_{Net physiological growth} + {\underbrace{{K}_{d} {N}_{live} ( t )}}_{death} = ( {K}_{d} + {K}_{g} [ 1 - \frac{{N}_{live} ( t )}{{N}_{max}} ] ) {N}_{live} ( t ) = ( {K}_{b} - {K}_{g} \frac{{N}_{live} ( t )}{{N}_{max}} ) {N}_{live} ( t ) \\ Eqns . ( 10 ) , ( 11 ) , and ( 13 ) \qquad ( 13 )$$

$$\frac{d {N}_{live}}{dt} = ( {K}_{g} [ 1 - \frac{{N}_{live} ( t )}{{N}_{max}} ] - {r}_{min} - λ a {e}^{- at} ) {N}_{live} ( t ) = ( - \frac{{N}_{live} ( t )}{{N}_{max} / {K}_{g}} + {K}_{g} - {r}_{min} - λ a {e}^{- at} ) {N}_{live} ( t ) \qquad ( 14 )$$

$$\frac{{dN}_{total}}{dt} = ( {K}_{g} [ 1 - \frac{{N}_{live} ( t )}{{N}_{max}} ] + {K}_{d} ) {N}_{live} ( t ) = ( - \frac{{N}_{live} ( t )}{{N}_{max} / {K}_{g}} + {K}_{b} ) {N}_{live} ( t ) \qquad ( 15 )$$

$$Eqn . ( 1 ) implies \frac{{dN}_{live}}{dt} = {K}_{g} [ 1 - \frac{{N}_{live} ( t )}{{N}_{max}} ] {N}_{live} ( t ) {N}_{live} ( t ) = {N}_{0} \text{?} \qquad ( 17 )$$

$$Eqns . ( 14 ) , ( 15 ) , and ( 17 ) imply \frac{{dN}_{total}}{dt} = ( {K}_{g} [ 1 - \frac{{N}_{live} ( t )}{{N}_{max}} ] + {K}_{d} ) {N}_{live} ( t )$$

$$\text{?} \qquad ( 18 )$$

$$\text{?} \text{indicates text missing or illegible when filed}$$

$$t \approx 0 \to$$

$$\text{?} \qquad ( 19 )$$

$$t \to ∞ \to$$

$${N}_{total} ( t ) \approx {N}_{max} {K}_{d} t \qquad ( 20 )$$

$$\text{?} \text{indicates text missing or illegible when filed}$$

$$Eqn . ( 1 ) \to$$

$$\text{?} \qquad ( 21 )$$

$$and$$

$$\text{?}$$

$$\text{?} \qquad ( 22 )$$

$$\text{?} \text{indicates text missing or illegible when filed}$$

$$\text{?} \qquad ( 23 )$$

$$\text{?}$$

$$\text{?} \qquad ( 24 )$$

$$\text{?} \text{indicates text missing or illegible when filed}$$

### APPENDIX B

### Example 2

Connection Between K_{g}, K_{b}, and K_{d}
In the absence of an antibiotic, the growth dynamics of a bacterial population is characterized by

$$\frac{d {N}_{live}}{d t} = {\underbrace{{K}_{b} [ 1 - \frac{{N}_{live} ( t )}{{N}_{c}} ] {N}_{live} ( t )}}_{Physiological birth} - {\underbrace{{K}_{d} {N}_{live} ( t )}}_{Physiological death} = {\underbrace{( {K}_{b} - {K}_{d} )}}_{{K}_{g}} [ 1 - \frac{{K}_{b} {N}_{live} ( t )}{{K}_{b} - {K}_{d} {N}_{c}} ] {N}_{live} ( t ) = {\underbrace{{K}_{g} [ 1 - \frac{{N}_{live} ( t )}{{N}_{max}} ] {N}_{live} ( t )}}_{Net physiological growth} where \qquad Eq . ( A \text{.1} )$$

$${N}_{max} = {N}_{c} \frac{{K}_{b} - {K}_{d}}{{K}_{b}} = {N}_{c} \frac{{K}_{g}}{{K}_{b}} \\ and \qquad Eq . ( A \text{.2} )$$

$${K}_{g} = {K}_{b} - {K}_{d} \qquad Eq . ( A \text{.3} )$$

### APPENDIX C

### Derivation of Eqn. (19)

Eqn. (12) can be solved analytically to yield

$$\frac{{N}_{live} ( t )}{{N}_{0}} = \frac{{e}^{λ ( {e}^{- a t} - 1 ) + ( {K}_{g} - {r}_{min} ) t}}{1 + {K}_{g} \frac{{N}_{0}}{{N}_{max}} {\int }_{0}^{t} {e}^{λ ( {e}^{- a τ} - 1 ) + ( {K}_{g} - {r}_{min} ) τ} d τ} = \frac{{e}^{λ ( {e}^{- a t} - 1 ) + ( {K}_{g} - {r}_{min} ) t}}{1 + {K}_{g} \frac{{N}_{0} {e}^{- λ}}{{N}_{max} a} {λ}^{\frac{{K}_{g} - {r}_{min}}{a}} {\int }_{λ {e}^{- at}}^{λ} {z}^{- 1 + \frac{{r}_{min} - {K}_{g}}{a}} {e}^{z} d z} with λ > 0 , a > 0 , {r}_{min} > 0. Therefore \\ \frac{{dN}_{total}}{d t} = \frac{{dN}_{live}}{d t} + \frac{d {N}_{d e a d}}{d t} ⇒ \frac{{N}_{total} ( t )}{{N}_{0}} = \frac{{N}_{live} ( t )}{{N}_{0}} + {\int }_{0}^{t} \frac{d {N}_{d e a d} / {N}_{0}}{d τ} d τ = \frac{{N}_{live} ( t )}{{N}_{0}} + {\int }_{0}^{t} ( {K}_{d} + μ ( τ ) ) \frac{{N}_{live} ( τ )}{{N}_{0}} d τ \\ which implies eqn . ( 17 ) . \qquad Eq . ( B \text{.1} )$$

What is claimed:

1. A method for determining a clinical dosing regimen that is pharmacologically effective against a microbial cell population in a subject comprising;
(i) collecting information-rich datasets that indicate microbe cell population growth response in the presence of one or more antimicrobial agents over a period of time at fixed concentrations;
(ii) inputting said datasets into the mathematical modeling framework (1) for determining the susceptibility of the microbe cell population during contact with the one or more antimicrobial agents over the period of time
$$\{ \begin{matrix}\frac{{dN}_{total}}{d t} = {K}_{g} [ 1 - \frac{{N}_{live} ( t )}{{N}_{max}} ] + {K}_{d} ) {N}_{live} ( t ) \\ \frac{{dN}_{live}}{d t} = {K}_{g} [ 1 - \frac{{N}_{live} ( t )}{{N}_{max}} ] - {r}_{min} - λ {ae}^{- at} ) {N}_{live} ( t )\end{matrix} \} \qquad ( 1 )$$
wherein Nₜₒₜₐₗ is the total bacterial population; Nₗᵢᵥₑ is the bacterial population that is alive; Nₘₐₓ is the maximum bacterial population; K_{q} is the growth rate constant; K_{d} is the death rate constant; rₘᵢₙ is the kill rate of the most resistant sub-population; λ is the magnitude of adaptation; and a is the rate of adaptation; and
(iii) generating an output value of the susceptibility of the microbe cell population based on the mathematical modeling frame work; and
(iv) based on the generated output value, correlating, at the end of the time period, an increase in microbe susceptibility in the presence of the antimicrobial agent with a likely clinical dosing regimen that is pharmacologically effective against the microbial cell population in the subject.

2. The method of claim 1, further comprising designing a dosing regimen that is pharmacologically effective against the microbial cell population based on the output values over the time period of the mathematical modeling framework.

3. The method of claim 1, wherein the microbial cell population is a cell population of Gram-negative bacteria, Gram-positive bacteria, yeast, mold, mycobacteria, virus, or infectious agents.

4. The method of claim 1, wherein the antimicrobial agent is an antibiotic, an anti-fungal or anti-viral agent.

5. A method of treating a subject having a pathological condition caused by infection with a microbial cell population using the antimicrobial dosing regimen determined by the method of claim 1.

6. A method of preventing a pathological condition caused by exposure of a subject to a microbial cell population using the antimicrobial dosing regimen determined by the method of claim 1.

7. The method of claim 1, wherein the information-rich datasets that indicate microbe cell population growth response in the presence of one or more antimicrobial agents are optically derived.

8. A method for determining a clinical dosing regimen that is pharmacologically effective against a microbial cell population that has developed a resistance to one or more antimicrobial agents in a subject comprising:
(i) collecting information-rich datasets that indicate microbial cell population growth response in the presence of one or more antimicrobial agents over a period of time wherein said microbial cell population has developed resistance to one or more antimicrobial agents;
(ii) inputting said datasets into the mathematical modeling framework (1) for determining the susceptibility of the microbe cell population during contact with the one or more antimicrobial agents over the period of time
$$\{ \begin{matrix}\frac{{dN}_{total}}{d t} = {K}_{g} [ 1 - \frac{{N}_{live} ( t )}{{N}_{max}} ] + {K}_{d} ) {N}_{live} ( t ) \\ \frac{{dN}_{live}}{d t} = {K}_{g} [ 1 - \frac{{N}_{live} ( t )}{{N}_{max}} ] - {r}_{min} - λ {ae}^{- at} ) {N}_{live} ( t )\end{matrix} \} \qquad ( 1 )$$
wherein Nₜₒₜₐₗ is the total bacterial population; Nᵢᵢ is the bacterial population that is alive; Nₘₐₓ is the maximum bacterial population; K₉ is the growth rate constant; K_{d} is the death rate constant; rₘᵢₙ is the kill rate of the most resistant sub-population; λ is the magnitude of adaptation; and a is the rate of adaptation; and
(iii) generating an output value of the susceptibility of the microbe cell population based on the mathematical modeling frame work; and
(iv) based on the generated output value, correlating at the end of the time period, an increase in microbe susceptibility in the presence of the one or more antimicrobial agents with a likely clinical dosing regimen that is pharmacologically effective against a resistant microbial cell population in a subject.

9. The method of claim 8, further comprising designing a dosing regimen that is pharmacologically effective against the microbial cell population wherein the microbial cell population has developed a resistance to the one of more antimicrobial agents.

10. The method of claim 8, further comprising compiling a library of antimicrobial agents and dosing regimens effective to suppress an emergence of acquired resistance in microbial cell populations.

11. The method of claim 8, wherein the microbial cell population is a cell population of Gram-negative bacteria, Gram-positive bacteria, yeast, mold, mycobacteria, virus, or infectious agents.

12. The method of claim 8, wherein the antimicrobial agent is an antibiotic, an anti-fungal or anti-viral agent.

13. A method of treating a subject having a pathological condition caused by infection with a resistant microbial cell population using the antimicrobial dosing regimen determined by the method of claim 8.

14. A method of preventing a pathological condition caused by exposure of a subject to a resistant microbial cell population using the antimicrobial dosing regimen determined by the method of claim 8.

15. The method of claim 8, wherein the information-rich datasets that indicate microbe cell population growth response in the presence of one or more antimicrobial agents are optically derived.

16. A method for determining a clinical dosing regimen that is pharmacologically effective against a microbial cell population in a subject comprising;
(i) collecting information-rich datasets that indicate microbe cell population growth response in the presence of one or more antimicrobial agents over a period of time at fixed concentrations;
(ii) inputting said datasets into the mathematical modeling framework (2) for determining the susceptibility of the microbe cell population during contact with the one or more antimicrobial agents over the period of time
$$\frac{{N}_{total} ( t )}{{N}_{0}} = {e}^{λ ( {e}^{- a t} - 1 ) + ( {K}_{g} - {r}_{min} ) t} + + {e}^{- λ} {λ}^{\frac{{K}_{g} - {r}_{min}}{a}} ( \frac{{K}_{d} + {r}_{min}}{a} {\int }_{λ {e}^{- at}}^{λ} {z}^{- 1 + \frac{{r}_{min} - {K}_{g}}{a}} {e}^{z} dz + {\int }_{λ {e}^{- at}}^{λ} {z}^{\frac{{r}_{min} - {K}_{g}}{a}} {e}^{z} dz ) ; \qquad ( 2 )$$
(iii) generating an output value of the susceptibility of the microbe cell population based on the mathematical modeling frame work; and
(iv) based on the generated output value, correlating, at the end of the time period, an increase in microbe susceptibility in the presence of the antimicrobial agent with a likely clinical dosing regimen that is pharmacologically effective against the microbial cell population in the subject.

17. The method of claim 16, further comprising designing a dosing regimen that is pharmacologically effective against the microbial cell population based on the output values over the time period of the mathematical modeling framework.

18. The method of claim 16, wherein the microbial cell population is a cell population of Gram negative bacteria, Gram positive bacteria, yeast, mold, mycobacteria, virus, or infectious agents.

19. The method of claim 16, wherein the antimicrobial agent is an antibiotic, an anti-fungal or anti-viral agent.

20. A method of treating a subject having a pathological condition caused by infection with a microbial cell population using the antimicrobial dosing regimen determined by the method of claim 16.

21. A method of preventing a pathological condition caused by exposure of a subject to a microbial cell population using the antimicrobial dosing regimen determined by the method of claim 16.

22. The method of claim 16, wherein the information-rich datasets that indicate microbe cell population growth response in the presence of one or more antimicrobial agents are optically derived.

23. A method for determining a clinical dosing regimen that is pharmacologically effective against a microbial cell population that has developed a resistance to one or more antimicrobial agents in a subject comprising:
(i) collecting information-rich datasets that indicate microbial cell population growth response in the presence of one or more antimicrobial agents over a period of time wherein said microbial cell population has developed resistance to one or more antimicrobial agents;
(ii) inputting said datasets into the mathematical modeling framework (2) for determining the susceptibility of the microbe cell population during contact with the one or more antimicrobial agents over the period of time
$$\frac{{N}_{total} ( t )}{{N}_{0}} = {e}^{λ ( {e}^{- a t} - 1 ) + ( {K}_{g} - {r}_{min} ) t} + + {e}^{- λ} {λ}^{\frac{{K}_{g} - {r}_{min}}{a}} ( \frac{{K}_{d} + {r}_{min}}{a} {\int }_{λ {e}^{- at}}^{λ} {z}^{- 1 + \frac{{r}_{min} - {K}_{g}}{a}} {e}^{z} dz + {\int }_{λ {e}^{- at}}^{λ} {z}^{\frac{{r}_{min} - {K}_{g}}{a}} {e}^{z} dz ) ; \qquad ( 2 )$$
(iii) generating an output value of the susceptibility of the microbe cell population based on the mathematical modeling framework; and
(iv) based on the generated output value, correlating at the end of the time period, an increase in microbe susceptibility in the presence of the one or more antimicrobial agents with a likely clinical dosing regimen that is pharmacologically effective against a resistant microbial cell population in a subject.

24. The method of claim 23, further comprising designing a dosing regimen that is pharmacologically effective against the microbial cell population wherein the microbial cell population has developed a resistance to the one of more antimicrobial agents.

25. The method of claim 23, further comprising compiling a library of antimicrobial agents and dosing regimens effective to suppress an emergence of acquired resistance in microbial cell populations.

26. The method of claim 23, wherein the microbial cell population is a cell population of Gram-negative bacteria, Gram-positive bacteria, yeast, mold, mycobacteria, virus, or infectious agents.

27. The method of claim 23, wherein the antimicrobial agent is an antibiotic, an anti-fungal or anti-viral agent.

28. A method of treating a subject having a pathological condition caused by infection with a microbial cell population that has developed resistance using the antimicrobial dosing regimen determined by the method of claim 23.

29. The method of claim 23, wherein the information-rich datasets that indicate microbe cell population growth response in the presence of one or more antimicrobial agents are optically derived.
